(19) United States
(12) Patent Application Publication
(10) Pub. No.: US 2022/0347123 A1
(43) Pub. Date: Nov. 3, 2022

(54) COMBINATIONS OF GABA-A RECEPTOR POSITIVE ALLOSTERIC MODULATORS AND NMDA ANTAGONISTS, NMDA NEGATIVE ALLOSTERIC MODULATORS OR NMDA PARTIAL AGONISTS

(71) Applicant: Praxis Precision Medicines, Inc., Boston, MA (US)

(72) Inventors: Carlos LOYA, Cambridge, MA (US); Zoe HUGHES, Newton, MA (US); Kiran REDDY, Charlestown, MA (US)

(21) Appl. No.: 17/765,975

(22) PCT Filed: Oct. 2, 2020
(86) PCT No.: PCT/US2020/053951

§ 371 Date: Apr. 1, 2022

Related U.S. Application Data
(63) Continuation of application No. 62/909,323, filed on Oct. 2, 2019.

(51) Int. Cl. A61K 31/135; A61K 31/56; A61K 9/00

(52) U.S. Cl. CPC A61K 31/135; A61K 31/56; A61K 9/0019; A61K 9/0043; A61K 9/0053

(57) ABSTRACT

The invention relates to combinations of GABA-A Receptor positive allosteric modulators and NMDA antagonists, NMDA Negative Allosteric Modulators or NMDA partial agonists and methods using such combinations to treat mood disorders, such as depression and anxiety.

### CROSS REFERENCE TO RELATED APPLICATIONS

This application claims priority to U.S. Application No. 62/909,323, filed Oct. 2, 2019, which is hereby incorporated by reference in its entirety herein.

### FIELD OF THE DISCLOSURE

The present disclosure relates to combinations of GABA-A Receptor positive allosteric modulators and NMDA antagonists, NMDA Negative Allosteric Modulators or NMDA partial agonists and methods using such combinations to treat mood disorders, such as depression and anxiety.

### BACKGROUND OF THE DISCLOSURE

Major depressive disorder affects a significant portion of the population and, according to the World Health Organization, is the number one cause of disability worldwide. Many of the FDA-approved therapies for treating MDD (such as SSRIs and SNRIs) have a substantial delay in the onset of efficacy (several weeks) and high rates of treatment failure, e.g., about 33% of MDD patients fail to achieve full symptomatic remission despite multiple treatment regimens. The lack of efficacy and delayed onset of efficacy of current medications are particularly problematic for a patient population at elevated risk for suicide. Thus, there is a need for fast acting, effective treatments for major depressive disorder.

3α-Hydroxy-3β-methoxymethyl-21-(1′-imidazolyl)-5α-pregnan-20-one (Compound 1) is a synthetic neuroactive steroid. Its primary molecular target is the γ-aminobutyric acid type A (GABA-A) receptor, where it acts as a positive allosteric modulator (PAM) of channel function. The structural formula of Compound 1 appears below.

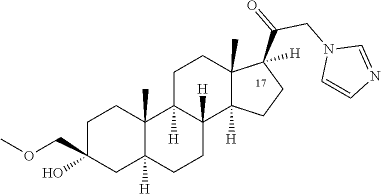

GABA-A Receptor positive allosteric modulators (GABA-A PAMs), especially neuroactive steroid GABA-A PAMs, have demonstrated clinical efficacy in anesthesia, epilepsy, post-partum depression, and major depression.

Some literature suggests that ketamine, an NMDA antagonist, may be used to treat certain forms of depression. However, there are no ketamine-containing drug products that are FDA-approved to treat depression.

### SUMMARY OF THE DISCLOSURE

The present disclosure, among other things, provides methods of treating depression (e.g., major depressive disorder, major depressive disorder with suicidal risk, clinical depression, postnatal or postpartum depression, treatment resistant postpartum depression, perimenopausal depression, premenstrual dysphoric disorder (PMDD), atypical depression, melancholic depression, Psychotic Major Depression (PMD), catatonic depression, Seasonal Affective Disorder (SAD), persistent depressive disorder (dysthymia), double depression, Depressive Personality Disorder (DPD), Recurrent Brief Depression (RBD), minor depressive disorder, bipolar disorder or manic depressive disorder, bipolar depression with suicidal risk, post-traumatic stress disorders, depression caused by chronic medical conditions, depressive disorder due to another medical condition, treatment-resistant depression, refractory depression, substance/medication induced depressive disorder, depression with anxiety, suicidality, suicidal ideation, or suicidal behavior) by administering a therapeutically effective amount of a GABA-A PAM (e.g., a neuroactive steroid) and a therapeutically effective amount of a NMDA antagonist, NMDA Negative Allosteric Modulator or NMDA partial agonist to a patient in need thereof. In another aspect, the present invention provides methods of treating a mood or affective disorder selected from perimenopause, generalized anxiety disorder, panic disorder, social anxiety disorder, post-traumatic stress disorder, specific phobia, and selective mutism by administering a therapeutically effective amount of a GABA-A PAM and a therapeutically effective amount of a NMDA antagonist, NMDA Negative Allosteric Modulator or NMDA partial agonist to a patient in need thereof.

In some embodiments, the patient in need of a treatment of depression is a patient with major depressive disorder (MDD). In some embodiments, the patient has severe MDD. In some embodiments, the patient in need of a treatment of depression is a patient with depression is refractory to other therapies (i.e., treatment resistant depression).

In one aspect, the present disclosure provides methods of pain by administering a therapeutically effective amount of a GABA-A PAM (e.g., a neuroactive steroid) and a therapeutically effective amount of a NMDA antagonist, NMDA Negative Allosteric Modulator or NMDA partial agonist to a patient in need thereof.

In one aspect, the present disclosure provides pharmaceutical compositions containing a therapeutically effective amount of a GABA-A PAM and therapeutically effective amount of a NMDA antagonist, NMDA Negative Allosteric Modulator or NMDA partial agonist. In another aspect, the present disclosure provides pharmaceutical kits containing a pharmaceutical composition containing a therapeutically effective amount of a GABA-A PAM and a pharmaceutical composition containing a therapeutically effective amount of a NMDA antagonist, NMDA Negative Allosteric Modulator or NMDA partial agonist. The kits and compositions provided by the present disclosure are useful for the treatment of mood and affective disorders.

In some embodiments of the present disclosure, the GABA-A PAM is Compound 1 or a pharmaceutically acceptable salt thereof and the NMDA antagonist is ketamine or a pharmaceutically acceptable salt thereof.

In some embodiments of the present disclosure, the GABA-A PAM is Compound 1 or a pharmaceutically acceptable salt thereof and the NMDA antagonist is esketamine or a pharmaceutically acceptable salt thereof.

### Definitions

The term “about” when immediately preceding a numerical value means a range (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example in a list of numerical values such as “about 49, about 50, about 55, . . . ”, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range.

Throughout this disclosure, various patents, patent applications and publications are referenced. The disclosures of these patents, patent applications and publications in their entireties are incorporated into this disclosure by reference for all purposes in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

The terms “administer,” “administering” or “administration” as used herein refer to either directly administering a compound or pharmaceutically acceptable salt or ester of the compound or a composition comprising the compound or pharmaceutically acceptable salt or ester of the compound to a patient.

The term “carrier” as used herein encompasses carriers, excipients, and diluents, meaning a material, composition or vehicle, such as a liquid or solid filler, diluent, excipient, solvent or encapsulating material involved in carrying or transporting a pharmaceutical agent from one organ, or portion of the body, to another organ or portion of the body.

The term “major depressive disorder” is used in this disclosure to mean major depressive disorder as defined in Diagnostic and Statistical Manual of Mental Disorders (DSM-5). The term “moderate major depressive disorder” is used in this disclosure to mean major depressive disorder where the number of symptoms, intensity of symptoms, and/or functional impairment are between those specified in the DSM-5 for “mild” and “severe.” The term “severe major depressive disorder” is used in this disclosure to mean major depressive disorder where the number of symptoms is substantially in excess of that required to make the diagnosis, the intensity of the symptoms is seriously distressing and unmanageable, and the symptoms markedly interfere with social and occupational functioning.

The term “disorder” is used in this disclosure to mean, and is used interchangeably with, the terms disease, condition, or illness, unless otherwise indicated.

The terms “effective amount” and “therapeutically effective amount” are used interchangeably in this disclosure and refer to an amount of a compound, or a salt, solvate or ester thereof, that, when administered to a patient, is capable of performing the intended result. For example, an effective amount of a GABA-A PAM (or an NMDA antagonist) is that amount that is required to reduce at least one symptom of depression in a patient. The actual amount that comprises the “effective amount” or “therapeutically effective amount” will vary depending on a number of conditions including, but not limited to, the severity of the disorder, the size and health of the patient, and the route of administration. A skilled medical practitioner can readily determine the appropriate amount using methods known in the medical arts.

The phrase “perimenopause” as used herein refers to early and late menopause transition stages as well as early postmenopause.

The phrase “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “salts” as used herein embraces pharmaceutically acceptable salts commonly used to form addition salts of free bases. The nature of the salt is not critical, provided that it is pharmaceutically acceptable. The term “salts” also includes solvates of addition salts, such as hydrates, as well as polymorphs of addition salts. Suitable pharmaceutically acceptable acid addition salts can be prepared from an inorganic acid or from an organic acid.

The term “treating” as used herein with regard to a patient, refers to improving at least one symptom of the patient's disorder. Treating can be curing, improving, or at least partially ameliorating a disorder.

The term “therapeutic effect” as used herein refers to a desired or beneficial effect provided by the method and/or the composition. For example, the method for treating depression provides a therapeutic effect when the method reduces at least one symptom of depression in a patient.

### DETAILED DESCRIPTION OF THE DISCLOSURE

Major Depressive Disorder (MDD) is a common psychiatric illness that causes disability and diminishes quality of life, depletes limited health care resources, increases morbidity and mortality, and increases the rates of substance abuse and suicide. The incidence of MDD in the United States and Australia is about 7% (American Psychiatric Association. (2013). Diagnostic and statistical manual of mental disorders (5th ed.). Arlington, Va.: American Psychiatric Publishing) and 5% (Australian Bureau of Statistics. (2008). National Survey of Mental Health and Wellbeing: Summary of results, 2007, cat no. 4326.0. Canberra: ABS), respectively. Current treatment options for MDD patients are limited and include serotonin reuptake inhibitors (SSRIs) and serotonin norepinephrine uptake inhibitors (SNRIs). However, SSRIs and SNRIs have serious liabilities in the context of treating MDD, such as a substantial delay in the onset of efficacy (several weeks) and high rates of treatment failure, e.g., about 33% of MDD patients fail to achieve full symptomatic remission despite multiple treatment regimens (Rush A J, et al. Acute and longer-term outcomes in depressed outpatients requiring one or several treatment steps: a STAR*D report. Am J Psychiatry. 2006; 163:1905-1917).

Disturbances of the GABAergic system have been implicated in the development of depression and anxiety. A growing body of preclinical and clinical evidence supports the hypothesis that GABA-A hypofunction plays a role in the pathophysiology of depression and anxiety (Luscher B, Shen Q, Sahir N. The GABAergic deficit hypothesis of major depressive disorder. Mol Psychiatry. 2011; 16 (4):383-406). Supporting this hypothesis, drugs that enhance GABAergic function have shown some clinical benefit in the treatment of mood disorders. For example, benzodiazepines, which are positive allosteric modulators (PAMs) of the GABA-A receptor, are highly efficacious in the treatment of anxiety disorders.

Neuroactive steroids (NASs) are a family of compounds (synthetic and naturally occurring) that affect neurophysiological functions through allosteric modulation of GABA-A receptors. The endogenous NASs allopregnanolone and pregnanolone are GABA-A PAMs that are dysregulated in mood disorders and show preclinical efficacy in animal models of anxiety and depression. NASs bind to a different binding site on the GABA-A receptor than benzodiazepines or the endogenous agonist GABA (Hosie A M, Wilkins M E, Da Silva HMA, Smart TG. Endogenous neurosteroids regulate GABA-A receptors through two discrete transmembrane sites. Nature. 2006; 444(7118):486-489). Benzodiazepines exclusively potentiate GABA-A receptors that contain a gamma subunit, which are primarily localized at synapses. In contrast, NASs bind to alpha and beta subunits, which are present in a larger proportion of GABA-A receptors, resulting broad activity at both synaptic and extrasynaptic sites.

The rapid antidepressant action of the glutamatergic N-methyl-D-aspartate (NMDA) receptor antagonist ketamine has been shown clinically at doses of 0.1, 0.5, and 0.75 mg/kg IV. Ketamine is most commonly administered in the dose of 0.5 mg/kg IV, administered across 40 min infusion sessions. Ketamine is a racemic mixture of two enantiomers, S(+)-ketamine and R(−)-ketamine. Ketamine causes dissociative anesthesia in high doses and can be used as a general anesthetic. However, there is no ketamine-containing drug product that is approved by the U.S. Food and Drug Administration for the treatment of depression.

In one aspect, the present invention provides a method of treating depression comprising administering an effective amount of a GABA-A Receptor Positive Allosteric Modulator and an effective amount of an NMDA antagonist, NMDA Negative Allosteric Modulator or NMDA partial agonist to a patient in need of such treatment. In another aspect, the present invention provides a method of treating a mood or affective disorder selected from perimenopause, generalized anxiety disorder, panic disorder, social anxiety disorder, post-traumatic stress disorder, specific phobia, and selective mutism comprising administering an effective amount of a GABA-A Receptor Positive Allosteric Modulator and an effective amount of an NMDA antagonist, NMDA Negative Allosteric Modulator or NMDA partial agonist to a patient in need of such treatment.

In accordance with some embodiments of the present invention, the GABA-A Receptor Positive Allosteric Modulator is Compound 1 or a pharmaceutically acceptable salt thereof and the NMDA antagonist is ketamine or a pharmaceutically acceptable salt thereof.

In accordance with some embodiments of the present invention, the GABA-A Receptor Positive Allosteric Modulator is Compound 1 or a pharmaceutically acceptable salt thereof and the NMDA antagonist is esketamine or a pharmaceutically acceptable salt thereof.

### GABA-A Receptor Positive Allosteric Modulators

According to the present disclosure, one or more GABA-A Receptor Positive Allosteric Modulators (GABA-A PAMs) are combined with one or more NMDA antagonists, NMDA Negative Allosteric Modulators or NMDA partial agonists to provide compositions and kits that are used to treat mood and affective disorders. In some embodiments, one or more GABA-A PAMs are combined with one or more NMDA antagonists, NMDA Negative Allosteric Modulators or NMDA partial agonists to provide compositions and kits that are used to treat pain.

GABA-A PAMs are known to those skilled in the art. The following disclosure provides guidance for the selection of GABA-A PAMs as well as non-limiting examples of GABA-A PAMs that are suitable for use in the compositions, kits and methods of the present disclosure.

GABA-A PAMs as employed in the present methods can form a part of a pharmaceutical composition by combining a GABA-A PAM, or a pharmaceutically acceptable salt thereof, with a pharmaceutically acceptable carrier. Additionally, the compositions can include an additive selected from the group consisting of adjuvants, excipients, diluents, release-modifying agents and stabilizers. The composition can be an immediate release formulation, a delayed release formulation, a sustained release formulation or an extended release formulation.

A suitable GABA-A PAM for use in the compositions, kits and methods of the present disclosure is a compound that: is a potent (<3 μM) modulator of synaptic and/or extrasynaptic GABA-A receptors, exhibits synergistic effects when administered in combination with NMDA antagonists, NMDA Negative Allosteric Modulators or NMDA partial agonists, and prevents or reverses a depression and/or anxiety phenotype in pre-clinical models of depression. In some embodiments, when administered in combination with a NMDA antagonist, a suitable GABA-A PAM does not worsen and potentially mitigates the side effect profile of ketamine and/or other NMDA antagonists, NMDA Negative Allosteric Modulators or NMDA partial agonists (e.g. decreases anxiety and/or dissociative signs).

In some embodiments, the GABA-A PAM is selected from the group consisting of benzodiazepines, neuroactive steroids, PF-06372865 (7-ethyl-4-[3-(4-ethylsulfonyl-2-methoxyphenyl)-4-fluorophenyl]imidazo[4,5-c]pyridazine), gaboxadol and etifoxine.

In some embodiments, the GABA-A PAM is a benzodiazepine. In some embodiments, the benzodiazepine is selected from the group consisting of midazolam, diazepam, chlordiazepoxide, alprazolam and adinazolam.

In some embodiments, the GABA-A PAM is a neuroactive steroid. In some embodiments, the neuroactive steroid is selected from the group consisting of pregnanolone, allopregnanolone, allotetrahydrodeoxycorticosterone, ganaxolone, alphaxolone, alphadolone, hydroxydione, minaxolone, Althesin, Renanolone, SAGE-324, SAGE-217 (3α-hydroxy-3β-methyl-21-(4-cyano-1H-pyrazol-1′-yl)-19-nor-5β-pregnan-20-one), and any neuroactive steroid as described in U.S. Publication No. 2017/0240589, which is hereby incorporated by reference in its entirety.

In preferred embodiments, the GABA-A PAM is 3α-Hydroxy-3β-methoxymethyl-21-(1′-imidazolyl)-5α-pregnan-20-one (Compound 1) or a pharmaceutically acceptable salt thereof. The structural formula of Compound 1 appears below.

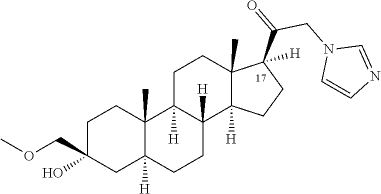

Compound 1 is a neuroactive steroid GABA-A PAM with high potency similar to clinical stage neuroactive steroids (allopregnanolone, ganaxolone, SAGE-217, alphaxolone).

The synthesis of Compound 1 is described in U.S. Publication Nos. 2004/034002 and 2009/0118248; crystalline polymorph of Compound 1 free base is described in U.S. Publication No. 2006/0074059; pharmaceutical compositions containing Compound 1 are described in U.S. Publication No. 2009/0131383, which are hereby incorporated by reference in their entirety for all purposes.

In some embodiments, the GABA-A PAM used in the formulations and methods of the present disclosure is Compound 1 or a pharmaceutically acceptable salt thereof. Salts of Compound 1 and polymorphs thereof are described in U.S. Provisional Application No. 62/725,805, which is hereby incorporated by reference in its entirety. In some embodiments, the pharmaceutically acceptable salt of Compound 1 used in the formulations and methods of the present disclosure is selected from the group consisting of hydrobromide, citrate, malate, mesylate, phosphate, tartrate, hydrochloride, tosylate, glucuronate, ethanesulfonate, fumarate, sulfate, napthalene-2-sulfonate, ascorbate, oxalate, napthalene-1,5-disulfonate, malonate, aminosalicylate, benzenesulfonate, isethionate, gentisate, 1-hydroxy-2-napthoate, dichloroacetate, cyclamate, and ethane-1,2-disulfonate salts. In some embodiments, the salt of Compound 1 is Compound 1 Hydrobromide. In some embodiments, the salt of Compound 1 is Compound 1 Citrate. In some embodiments, the salt of Compound 1 is Compound 1 L-Malate. In some embodiments, the salt of Compound 1 is Compound 1 Mesylate. In some embodiments, the salt of Compound 1 is Compound 1 Phosphate. In some embodiments, the salt of Compound 1 is Compound 1 L(+)-Tartrate. In some embodiments, the salt of Compound 1 is Compound 1 Hydrochloride. In some embodiments, the salt of Compound 1 is Compound 1 Tosylate. In some embodiments, the salt of Compound 1 is Compound 1 Glucuronate. In some embodiments, the salt of Compound 1 is Compound 1 Ethanesulfonate.

### NMDA Antagonists/NMDA Negative Allosteric Modulators/NMDA Partial Agonists/NMDA Open Channel Blocker:

According to the present disclosure, one or more NMDA antagonists, an NMDA open channel blockers, NMDA negative allosteric modulators or NMDA partial agonists are combined with one or more GABA-A PAMs to provide compositions and kits that are used to treat mood and affective disorders. In some embodiments, one or more NMDA antagonists, an NMDA open channel blockers, NMDA negative allosteric modulators or NMDA partial agonists are combined with one or more GABA-A PAMs to provide compositions and kits that are used to treat pain.

NMDA antagonists, an NMDA open channel blockers, NMDA negative allosteric modulators or NMDA partial agonists are known to those skilled in the art. The following disclosure provides guidance for the selection of NMDA antagonists, an NMDA open channel blockers, NMDA negative allosteric modulators or NMDA partial agonists as well as non-limiting examples of NMDA antagonists, an NMDA open channel blockers, NMDA negative allosteric modulators or NMDA partial agonists that are suitable for use in the compositions, kits and methods of the present disclosure.

NMDA antagonists, NMDA open channel blockers, NMDA negative allosteric modulators or NMDA partial agonists as employed in the present methods can form a part of a pharmaceutical composition by combining NMDA antagonists, NMDA negative allosteric modulators and NMDA partial agonists or a pharmaceutically acceptable salt thereof, with a pharmaceutically acceptable carrier. Additionally, the compositions can include an additive selected from the group consisting of adjuvants, excipients, diluents, release-modifying agents and stabilizers. The composition can be an immediate release formulation, a delayed release formulation, a sustained release formulation or an extended release formulation.

A suitable NMDA Negative allosteric modulator (NAM) for use in the compositions, kits and methods of the present disclosure is a compound that binds to an allosteric binding site on the NMDA receptor and reduces the activity of the NMDA receptor in response to the endogenous co-agonists glutamate and glycine.

A suitable NMDA orthosteric (competitive) antagonist for use in the compositions, kits and methods of the present disclosure is a compound that binds to either the glutamate binding site or the glycine co-agonist binding site and reduces the activity of the NMDA receptor in response to the endogenous co-agonists glutamate and glycine.

A suitable NMDA receptor partial agonist for use in the compositions, kits and methods of the present disclosure is a compound that binds to the orthosteric (i.e. glutamate or glycine binding sites) or allosteric sites of the NMDA receptor and partially activates the receptor independent of endogenous agonist binding.

A suitable open channel blocker, is a compound that binds to a binding site that is located in the central pore of the channel that is not accessible unless the channel is in the open, activated state.

In some embodiments, the NMDA antagonist or NMDA negative allosteric modulator is selected from the group consisting of ketamine, R-ketamine, esketamine, methadone, D-methadone, BMT-108908 ((R)-1-(4-fluorobenzyl)-3-(4-(4-hydroxyphenyl)piperidin-1-yl)pyrrolidin-2-one), flupirtine radiprodil, priralfinamide dextromethorphan, AZD-8108, AZD-6423, NYX-2925 (((2S, 3R)-3-hydroxy-2-((R)-5-isobutyryl-1-oxo-2,5-diazaspiro[3,4]octan-2-yl)butanamide)), NYX-783, NYX-458, lanicemine, AZD 6423, traxoprodil, ifenprodil, rislenemdaz (CERC-301), EVT-101 (5-(3-(difluoromethyl)-4-fluorophenyl)-3-((2-methyl-1H-imidazol-1-yl)methyl)pyridazine), 4-chlorokynurenine, memantine, amantadine, methoxetamine, dextropropoxyphene, ketobemidone, phencyclidine, and riluzole.

In some embodiments, the NMDA antagonist or NMDA negative allosteric modulator is selected from the group consisting of ketamine, R-ketamine, esketamine, methadone, D-methadone, BMT-108908 ((R)-1-(4-fluorobenzyl)-3-(4-(4-hydroxyphenyl)piperidin-1-yl)pyrrolidin-2-one), flupirtine radiprodil, priralfinamide dextromethorphan, lanicemine, traxoprodil, ifenprodil, rislenemdaz (CERC-301), EVT-101 (5-(3-(difluoromethyl)-4-fluorophenyl)-3-((2-methyl-1H-imidazol-1-yl)methyl)pyridazine), dextropropoxyphene, ketobemidone, phencyclidine, and memantine.

In some embodiments, the NMDA antagonist is ketamine or a pharmaceutically acceptable salt thereof. In some embodiments, the NMDA antagonist is ketamine hydrochloride.

In some embodiments, the NMDA antagonist is esketamine or a pharmaceutically acceptable salt thereof. In some embodiments, the NMDA antagonist is esketamine hydrochloride.

In some embodiments, the NMDA partial agonist selected from the group consisting of Rapastinel, AGN-241751, and d-cycloserine.

### Formulations/Kits

The methods of the present invention can employ various formulations for administration to patients, e.g., humans in unit dosage forms, such as tablets, capsules, pills, powders, granules, sterile parenteral solutions or suspensions (e.g., intramuscular (IM), subcutaneous (SC) and intravenous (IV)), transdermal patches, and oral solutions or suspensions, and oil-water emulsions containing suitable quantities of the GABA-A PAMs and/or NMDA antagonists, NMDA Negative Allosteric Modulators or NMDA partial agonists of the present disclosure.

Oral pharmaceutical dosage forms can be either solid or liquid. A non-limiting list of suitable solid dosage forms includes tablets, capsules, granules, films (e.g., buccal films) and bulk powders. Types of oral tablets include, for example, compressed, chewable lozenges and tablets, which can be enteric-coated, sugarcoated or film-coated. Capsules can be hard or soft gelatin capsules, while granules and powders can be provided in non-effervescent or effervescent form with the combination of other ingredients known to those skilled in the art. In some embodiments, the present oral dosage forms may include orally disintegrating tablets.

Pharmaceutically acceptable carriers utilized in tablets include binders, lubricants, diluents, disintegrating agents, coloring agents, flavoring agents, and wetting agents.

Liquid oral dosage forms include aqueous solutions, emulsions, suspensions, solutions and/or suspensions reconstituted from non-effervescent granules and effervescent preparations reconstituted from effervescent granules. Liquid nasal dosage forms include aqueous solution and suspensions.

Aqueous solutions include, for example, elixirs and syrups. Emulsions can be either oil-in water or water-in-oil. Elixirs are clear, sweetened, hydroalcoholic preparations. Pharmaceutically acceptable carriers used in elixirs include solvents. Syrups can be concentrated aqueous solutions of a sugar, for example, sucrose, and can contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions can use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substance used in effervescent granules, to be reconstituted into a liquid oral dosage form, can include organic acids and a source of carbon dioxide. Coloring and flavoring agents can be used in all of the above dosage forms.

In one aspect, the present disclosure provides compositions for treating a mood or affective disorder comprising: (a) a therapeutically effective amount of a GABA-A PAM and (b) a therapeutically effective amount of an NMDA antagonist, NMDA Negative Allosteric Modulator or NMDA partial agonist. In such embodiments, the GABA-A PAM and the NMDA antagonist, NMDA Negative Allosteric Modulator or NMDA partial agonist are present in the same composition.

In some embodiments, the present disclosure provides compositions where the GABA-A PAM is Compound 1:

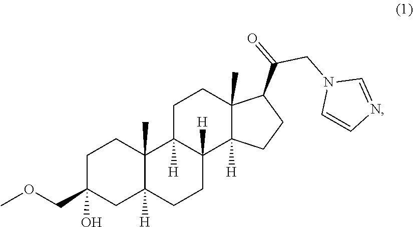

or a pharmaceutically acceptable salt thereof and the NMDA antagonist is ketamine or a pharmaceutically acceptable salt thereof.

In some embodiments, the compositions of the present disclosure contain from about 5 mg to about 120 mg of Compound 1 or a pharmaceutically acceptable salt thereof, including about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, and about 120 mg, including all ranges there between.

In some embodiments, the compositions of the present disclosure contain about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, or about 120 mg of Compound 1 or a pharmaceutically acceptable salt thereof.

In some embodiments, the compositions of the present disclosure contain from about 1 mg to about 250 mg of ketamine or a pharmaceutically acceptable salt thereof, including about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, or about 250 mg, including all ranges there between.

In some embodiments, the compositions of the present disclosure contain about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, or about 250 mg of ketamine or a pharmaceutically acceptable salt thereof.

In some embodiments, the compositions of the present disclosure comprising Compound 1 or a pharmaceutically acceptable salt thereof and ketamine or a pharmaceutically acceptable salt thereof is an oral dosage form. In some embodiments, the compositions of the present disclosure comprising Compound 1 or a pharmaceutically acceptable salt thereof and ketamine or a pharmaceutically acceptable salt thereof is a parenteral dosage form. In some embodiments, the compositions of the present disclosure comprising Compound 1 or a pharmaceutically acceptable salt thereof and ketamine or a pharmaceutically acceptable salt thereof is a subcutaneous dosage form. In some embodiments, the compositions of the present disclosure comprising Compound 1 or a pharmaceutically acceptable salt thereof and ketamine or a pharmaceutically acceptable salt thereof is an intramuscular dosage form. In some embodiments, the compositions of the present disclosure comprising Compound 1 or a pharmaceutically acceptable salt thereof and ketamine or a pharmaceutically acceptable salt thereof is an intranasal dosage form.

In some embodiments, upon administration to a patient in need thereof the compositions of the present disclosure provide different release rates for the GABA-A PAM and the NMDA antagonist, NMDA Negative Allosteric Modulator or NMDA partial agonist from the composition. In some embodiments, upon administration to a patient in need thereof the NMDA antagonist, NMDA Negative Allosteric Modulator or NMDA partial agonist is immediately released from the composition and the GABA-A PAM is released at least about 6 hours after administration (i.e., GABA-A PAM is delayed release and NMDA antagonist, NMDA Negative Allosteric Modulator or NMDA partial agonist is immediate release). In certain further embodiments, after the delayed release period, the GABA-A PAM is released over a period of at least about 4 hours (i.e., the GABA-A PAM is extended release). In some embodiments, upon administration to a patient in need thereof, the compositions of the present disclosure provide therapeutically effective blood plasma levels (as described herein) of the NMDA antagonist, NMDA Negative Allosteric Modulator or NMDA partial agonist between about 1 to about 8 hours after administration and provide therapeutically effective blood plasma levels (as described herein) GABA-A PAM between about 8 to about 12 hours after administration. Fixed dose combination compositions suitable to achieve the aforementioned release profiles are known to skilled in the art and include multilayer tablets, multiparticulate formulations, segmented tablets, osmotic tablets, etc.

According to some embodiments of the present invention, administration of the compositions of the present invention to a patient in need thereof provides therapeutically effective blood plasma levels of Compound 1 for treating depression. According to other embodiments of the present invention, administration of the compositions and kits of the present invention to a patient in need thereof provides therapeutically effective blood plasma levels of ketamine for treating depression. Blood plasma levels of Compound 1 and ketamine may be expressed using pharmacokinetic parameters that are known to those skilled in the art, such as steady state plasma levels, AUC, Cmax and Cmin.

In some embodiments, upon administration to a patient in need thereof the compositions of the present disclosure provide mean steady state AUC₀₋₂₄ h (expressed in terms of ng*hr/mL) levels of Compound 1 that correlate to one or more statistically significant therapeutic effects. In some embodiments, the therapeutically effective mean steady state AUC₀₋₂₄ h levels of Compound 1 provided by the compositions of the present invention upon administration to a patient in need thereof range from about 50 ng*hr/mL to about 2300 ng*hr/mL, including about 50 ng*hr/mL, about 100 ng*hr/mL, about 150 ng*hr/mL, about 200 ng*hr/mL, about 250 ng*hr/mL, 300 ng*hr/mL, about 400 ng*hr/mL, about 500 ng*hr/mL, about 600 ng*hr/mL, about 700 ng*hr/mL, about 800 ng*hr/mL, about 900 ng*hr/mL, about 1000 ng*hr/mL, about 1100 ng*hr/mL, about 1200 ng*hr/mL, about 1300 ng*hr/mL, about 1400 ng*hr/mL, about 1500 ng*hr/mL, about 1600 ng*hr/mL, about 1700 ng*hr/mL, about 1800 ng*hr/mL, about 1900 ng*hr/mL, about 2000 ng*hr/mL, about 2100 ng*hr/mL, about 2200 ng*hr/mL and about 2300 ng*hr/mL, including all ranges there between. In some embodiments, upon administration to a patient in need thereof the compositions of the present disclosure provide a mean steady state AUC₀₋₂₄ h from about 500 ng*h/ml to about 2500 ng*h/ml of Compound 1. In other embodiments, the compositions of the present disclosure include compositions in which the mean steady state AUC₀₋₂₄ h of Compound 1 falls within 80% to 125% of any of the mean steady state AUC₀₋₂₄ h values for Compound 1 provided herein.

In some embodiments, upon administration to a patient in need thereof the compositions of the present disclosure provide steady state plasma Cmax levels of Compound 1 that correlate to one or more statistically significant therapeutic effects. In some embodiments, the therapeutically effective steady state plasma Cmax levels of Compound 1 provided by the compositions of the present invention upon administration to a patient in need thereof range from about 5 ng/mL to about 500 ng/mL, including about 5 ng/mL, about 10 ng/mL, about 20 ng/mL, about 30 ng/mL, about 40 ng/mL, about 50 ng/mL, about 60 ng/mL, about 70 ng/mL, about 80 ng/mL, about 90 ng/mL, about 100 ng/mL, about 110 ng/mL, about 120 ng/mL, about 130 ng/mL, about 140 ng/mL, about 150 ng/mL, about 160 ng/mL, about 170 ng/mL about 180 ng/mL, about 190 ng/mL, about 200 ng/mL, about 210 ng/mL, about 220 ng/mL, about 230 ng/mL, about 240 ng/mL, about 250 ng/mL, about 260 ng/mL, about 270 ng/mL about 280 ng/mL, about 290 ng/mL, about 300 ng/mL, about 310 ng/mL, about 320 ng/mL, about 330 ng/mL, about 340 ng/mL, about 350 ng/mL, about 360 ng/mL, about 370 ng/mL about 380 ng/mL, about 390 ng/mL, about 400 ng/mL, about 410 ng/mL, about 420 ng/mL, about 430 ng/mL, about 440 ng/mL, about 150 ng/mL, about 460 ng/mL, about 470 ng/mL about 480 ng/mL, about 490 ng/mL, about 400 ng/mL, and about 500 ng/mL, including all ranges there between. In some embodiments, upon administration to a patient in need thereof the compositions of the present disclosure provide a steady state blood plasma Cmax from about 50 ng/mL to about 400 ng/ml of Compound 1. In other embodiments, the compositions of the present disclosure include compositions in which the steady state blood plasma Cmax of Compound 1 falls within 80% to 125% of any of the steady state blood plasma Cmax values for Compound 1 provided herein.

In some embodiments, upon administration to a patient in need thereof the compositions of the present disclosure provide steady state plasma Cmax levels of Compound 1 that do not exceed 500 ng/mL. In some embodiments, the therapeutically effective steady state plasma Cmax levels of Compound 1 provided by the compositions of the present invention upon administration to a patient in need thereof do not exceed about 500 ng/mL, including less than about 500 ng/mL, less than about 475 ng/mL, less than about 450 ng/mL, than about 425 ng/mL, less than about 400 ng/mL, less than about 375 ng/mL, than about 350 ng/mL, less than about 325 ng/mL, and less than about 300 ng/mL.

In some embodiments, upon administration to a patient in need thereof the compositions of the present disclosure provide mean steady state AUC₀₋₆ h (expressed in terms of ng*hr/mL) levels of ketamine that correlate to one or more statistically significant therapeutic effects. In some embodiments, the therapeutically effective mean steady state AUC₀₋₆ h levels of ketamine provided by the compositions of the present invention upon administration to a patient in need thereof range from about 50 ng*hr/mL to about 500 ng*hr/mL, including about 50 ng*hr/mL, about 75 ng*hr/mL, about 100 ng*hr/mL, about 125 ng*hr/mL, about 150 ng*hr/mL, about 175 ng*hr/mL, about 200 ng*hr/mL, about 200 ng*hr/mL, about 225 ng*hr/mL, about 250 ng*hr/mL, about 275 ng*hr/mL, about 300 ng*hr/mL, about 320 ng*hr/mL, about 350 ng*hr/mL, about 375 ng*hr/mL, about 400 ng*hr/mL, about 425 ng*hr/mL, about 450 ng*hr/mL, about 475 ng*hr/mL, and about 500 ng*hr/mL, including all ranges there between. In other embodiments, the compositions of the present disclosure include compositions in which the steady state blood plasma AUC₀₋₆ h of ketamine that falls within 80% to 125% of any of the steady state blood plasma AUC₀₋₆ h values for ketamine provided herein.

In some embodiments, upon administration to a patient in need thereof the compositions of the present disclosure provide mean steady state AUC₀₋₆ h (expressed in terms of ng*hr/mL) levels of norketamine (a ketamine and esketamine metabolite) that correlate to one or more statistically significant therapeutic effects. In some embodiments, the therapeutically effective mean steady state AUC₀₋₆ h levels of norketamine provided by the compositions of the present invention upon administration to a patient in need thereof range from about 50 ng*hr/mL to about 500 ng*hr/mL, including about 50 ng*hr/mL, about 75 ng*hr/mL, about 100 ng*hr/mL, about 125 ng*hr/mL, about 150 ng*hr/mL, about 175 ng*hr/mL, about 200 ng*hr/mL, about 200 ng*hr/mL, about 225 ng*hr/mL, about 250 ng*hr/mL, about 275 ng*hr/mL, about 300 ng*hr/mL, about 320 ng*hr/mL, about 350 ng*hr/mL, about 375 ng*hr/mL, about 400 ng*hr/mL, about 425 ng*hr/mL, about 450 ng*hr/mL, about 475 ng*hr/mL, and about 500 ng*hr/mL, including all ranges there between. In other embodiments, the compositions of the present disclosure include compositions in which the steady state blood plasma AUC₀₋₆ h of norketamine that falls within 80% to 125% of any of the steady state blood plasma AUC₀₋₆ h values for norketamine provided herein.

In some embodiments, upon administration to a patient in need thereof the compositions of the present disclosure provide steady state plasma Cmax levels of ketamine that correlate to one or more statistically significant therapeutic effects. In some embodiments, the therapeutically effective steady state plasma Cmax levels of ketamine provided by the compositions of the present invention upon administration to a patient in need thereof range from about 5 ng/mL to about 400 ng/mL, including about 5 ng/mL, about 10 ng/mL, about 20 ng/mL, about 30 ng/mL, about 40 ng/mL, about 50 ng/mL, about 60 ng/mL, about 70 ng/mL, about 80 ng/mL, about 90 ng/mL, about 100 ng/mL, about 110 ng/mL, about 120 ng/mL, about 130 ng/mL, about 140 ng/mL, about 150 ng/mL, about 160 ng/mL, about 170 ng/mL about 180 ng/mL, about 190 ng/mL, about 200 ng/mL, about 210 ng/mL, about 220 ng/mL, about 230 ng/mL, about 240 ng/mL, about 250 ng/mL, about 260 ng/mL, about 270 ng/mL about 280 ng/mL, about 290 ng/mL, about 300 ng/mL, about 310 ng/mL, about 320 ng/mL, about 330 ng/mL, about 340 ng/mL, about 350 ng/mL, about 360 ng/mL, about 370 ng/mL about 380 ng/mL, about 390 ng/mL, and about 400 ng/mL, including all ranges there between. In other embodiments, the compositions of the present disclosure include compositions in which the steady state blood plasma Cmax of ketamine that falls within 80% to 125% of any of the steady state blood plasma Cmax values for ketamine provided herein.

In some embodiments, upon administration to a patient in need thereof the compositions of the present disclosure provide steady state plasma Cmax levels of norketamine (a ketamine and esketamine metabolite) that correlate to one or more statistically significant therapeutic effects. In some embodiments, the therapeutically effective steady state plasma Cmax levels of norketamine provided by the compositions of the present invention upon administration to a patient in need thereof range from about 5 ng/mL to about 400 ng/mL, including about 5 ng/mL, about 10 ng/mL, about 20 ng/mL, about 30 ng/mL, about 40 ng/mL, about 50 ng/mL, about 60 ng/mL, about 70 ng/mL, about 80 ng/mL, about 90 ng/mL, about 100 ng/mL, about 110 ng/mL, about 120 ng/mL, about 130 ng/mL, about 140 ng/mL, about 150 ng/mL, about 160 ng/mL, about 170 ng/mL about 180 ng/mL, about 190 ng/mL, about 200 ng/mL, about 210 ng/mL, about 220 ng/mL, about 230 ng/mL, about 240 ng/mL, about 250 ng/mL, about 260 ng/mL, about 270 ng/mL about 280 ng/mL, about 290 ng/mL, about 300 ng/mL, about 310 ng/mL, about 320 ng/mL, about 330 ng/mL, about 340 ng/mL, about 350 ng/mL, about 360 ng/mL, about 370 ng/mL about 380 ng/mL, about 390 ng/mL, and about 400 ng/mL, including all ranges there between. In other embodiments, the compositions of the present disclosure include compositions in which the steady state blood plasma Cmax of norketamine that falls within 80% to 125% of any of the steady state blood plasma Cmax values for norketamine provided herein.

In one aspect, the present disclosure provides a kit for treating a mood or affective disorder comprising (a) a composition comprising a therapeutically effective amount of a GABA-A PAM and (b) a composition comprising a therapeutically effective amount of an NMDA antagonist, NMDA Negative Allosteric Modulator or NMDA partial agonist. In such embodiments, the GABA-A PAM and the NMDA antagonist, NMDA Negative Allosteric Modulator or NMDA partial agonist are present in separate compositions.

In some embodiments, the present disclosure provides kits where the GABA-A PAM is Compound 1:

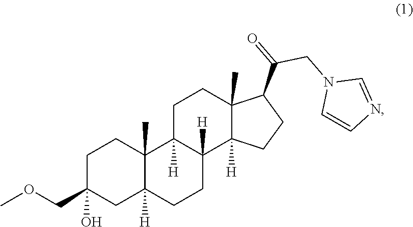

or a pharmaceutically acceptable salt thereof and the NMDA antagonist is ketamine or a pharmaceutically acceptable salt thereof.

In some embodiments, the kits of the present disclosure comprise a composition (a) containing from about 5 mg to about 120 mg of Compound 1 or a pharmaceutically acceptable salt thereof, including about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, and about 120 mg, including all ranges there between.

In some embodiments, the kits of the present disclosure comprise a composition (a) containing about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, or about 120 mg of Compound 1 or a pharmaceutically acceptable salt thereof.

In some embodiments, the kits of the present disclosure comprise a composition (b) containing from about 1 mg to about 250 mg of ketamine or a pharmaceutically acceptable salt thereof, including about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, or about 250 mg, including all ranges there between.

In some embodiments, the kits of the present disclosure comprise a composition (b) containing about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, or about 250 mg of ketamine or a pharmaceutically acceptable salt thereof.

In some embodiments of the kits of the present disclosure, the composition (b) is a nasal spray. In some embodiments of the kits of the present disclosure, the composition (b) is a solution suitable for intravenous administration. In some embodiments of the kits of the present disclosure, the composition (b) is a film suitable for sublingual administration.

In some embodiments, upon administration to a patient in need thereof the composition (a) of the present disclosure provide mean steady state AUC₀₋₂₄ h (expressed in terms of ng*hr/mL) levels of Compound 1 that correlate to one or more statistically significant therapeutic effects. In some embodiments, the therapeutically effective mean steady state AUC₀₋₂₄ h levels of Compound 1 provided by the composition (a) of the present invention range from about 50 ng*hr/mL to about 2300 ng*hr/mL, including about 50 ng*hr/mL, 100 ng*hr/mL, 150 ng*hr/mL, 200 ng*hr/mL, 250 ng*hr/mL, 300 ng*hr/mL, about 400 ng*hr/mL, about 500 ng*hr/mL, about 600 ng*hr/mL, about 700 ng*hr/mL, about 800 ng*hr/mL, about 900 ng*hr/mL, about 1000 ng*hr/mL, about 1100 ng*hr/mL, about 1200 ng*hr/mL, about 1300 ng*hr/mL, about 1400 ng*hr/mL, about 1500 ng*hr/mL, about 1600 ng*hr/mL, about 1700 ng*hr/mL, about 1800 ng*hr/mL, about 1900 ng*hr/mL, about 2000 ng*hr/mL, about 2100 ng*hr/mL, about 2200 ng*hr/mL and about 2300 ng*hr/mL, including all ranges there between. In some embodiments, upon administration to a patient in need thereof the composition (a) of the present disclosure provide a mean steady state AUC₀₋₂₄ h from about 500 ng*h/ml to about 2500 ng*h/ml of Compound 1. In other embodiments, the composition (a) of the kit of the present disclosure includes a composition in which the mean steady state AUC₀₋₂₄ h of Compound 1 falls within 80% to 125% of any of the mean steady state AUC₀₋₂₄ h values for Compound 1 provided herein.

In some embodiments, upon administration to a patient in need thereof the composition (a) of the present disclosure provide steady state plasma Cmax levels of Compound 1 that correlate to one or more statistically significant therapeutic effects. In some embodiments, the therapeutically effective steady state plasma Cmax levels of Compound 1 provided by the composition (a) of the present invention range from about 5 ng/mL to about 500 ng/mL, including about 5 ng/mL, 10 ng/mL, 20 ng/mL, 30 ng/mL, 40 ng/mL, 50 ng/mL, 60 ng/mL, about 70 ng/mL, about 80 ng/mL, about 90 ng/mL, about 100 ng/mL, about 110 ng/mL, about 120 ng/mL, about 130 ng/mL, about 140 ng/mL, about 150 ng/mL, about 160 ng/mL, about 170 ng/mL about 180 ng/mL, about 190 ng/mL, about 200 ng/mL, about 210 ng/mL, about 220 ng/mL, about 230 ng/mL, about 240 ng/mL, about 250 ng/mL, about 260 ng/mL, about 270 ng/mL about 280 ng/mL, about 290 ng/mL, about 300 ng/mL, about 310 ng/mL, about 320 ng/mL, about 330 ng/mL, about 340 ng/mL, about 350 ng/mL, about 360 ng/mL, about 370 ng/mL about 380 ng/mL, about 390 ng/mL, about 400 ng/mL, about 410 ng/mL, about 420 ng/mL, about 430 ng/mL, about 440 ng/mL, about 150 ng/mL, about 460 ng/mL, about 470 ng/mL about 480 ng/mL, about 490 ng/mL, about 400 ng/mL, and about 500 ng/mL, including all ranges there between. In some embodiments, upon administration to a patient in need thereof the composition (a) of the present disclosure provide a steady state blood plasma Cmax from about 50 ng/mL to about 400 ng/ml of Compound 1.

In some embodiments, upon administration to a patient in need thereof the composition (a) of the present disclosure provide steady state plasma Cmax levels of Compound 1 that do not exceed 500 ng/mL. In some embodiments, the therapeutically effective steady state plasma Cmax levels of Compound 1 provided by the composition (a) of the present invention do not exceed about 500 ng/mL, including less than about 500 ng/mL, less than about 475 ng/mL, less than about 450 ng/mL, than about 425 ng/mL, less than about 400 ng/mL, less than about 375 ng/mL, than about 350 ng/mL, less than about 325 ng/mL, and less than about 300 ng/mL. In other embodiments, the composition (a) of the kit of the present disclosure includes a composition in which the steady state plasma Cmax levels of Compound 1 falls within 80% to 125% of any of steady state plasma Cmax levels for Compound 1 provided herein.

In some embodiments, upon administration to a patient in need thereof the composition (b) of the present disclosure provides mean steady state AUC₀₋₆ h (expressed in terms of ng*hr/mL) levels of ketamine that correlate to one or more statistically significant therapeutic effects. In some embodiments, the therapeutically effective mean steady state AUC₀₋₆ h levels of ketamine provided by the composition (b) of the present invention upon administration to a patient in need thereof range from about 50 ng*hr/mL to about 500 ng*hr/mL, including about 50 ng*hr/mL, about 75 ng*hr/mL, about 100 ng*hr/mL, about 125 ng*hr/mL, about 150 ng*hr/mL, about 175 ng*hr/mL, about 200 ng*hr/mL, about 200 ng*hr/mL, about 225 ng*hr/mL, about 250 ng*hr/mL, about 275 ng*hr/mL, about 300 ng*hr/mL, about 320 ng*hr/mL, about 350 ng*hr/mL, about 375 ng*hr/mL, about 400 ng*hr/mL, about 425 ng*hr/mL, about 450 ng*hr/mL, about 475 ng*hr/mL, and about 500 ng*hr/mL, including all ranges there between. In other embodiments, the composition (b) of the present disclosure include compositions in which the steady state blood plasma AUC₀₋₆ h of ketamine that falls within 80% to 125% of any of the steady state blood plasma AUC₀₋₆ h values for ketamine provided herein.

In some embodiments, upon administration to a patient in need thereof the composition (b) of the present disclosure provide mean steady state AUC₀₋₆ h (expressed in terms of ng*hr/mL) levels of norketamine (a ketamine and esketamine metabolite) that correlate to one or more statistically significant therapeutic effects. In some embodiments, the therapeutically effective mean steady state AUC₀₋₆ h levels of norketamine provided by the composition (b) of the present invention upon administration to a patient in need thereof range from about 50 ng*hr/mL to about 500 ng*hr/mL, including about 50 ng*hr/mL, about 75 ng*hr/mL, about 100 ng*hr/mL, about 125 ng*hr/mL, about 150 ng*hr/mL, about 175 ng*hr/mL, about 200 ng*hr/mL, about 200 ng*hr/mL, about 225 ng*hr/mL, about 250 ng*hr/mL, about 275 ng*hr/mL, about 300 ng*hr/mL, about 320 ng*hr/mL, about 350 ng*hr/mL, about 375 ng*hr/mL, about 400 ng*hr/mL, about 425 ng*hr/mL, about 450 ng*hr/mL, about 475 ng*hr/mL, and about 500 ng*hr/mL, including all ranges there between. In other embodiments, the composition (b) of the present disclosure include compositions in which the steady state blood plasma AUC₀₋₆ h of norketamine that falls within 80% to 125% of any of the steady state blood plasma AUC₀₋₆ h values for norketamine provided herein.

In some embodiments, upon administration to a patient in need thereof the composition (b) of the present disclosure provide steady state plasma Cmax levels of ketamine that correlate to one or more statistically significant therapeutic effects. In some embodiments, the therapeutically effective steady state plasma Cmax levels of ketamine provided by the composition (b) of the present invention upon administration to a patient in need thereof range from about 5 ng/mL to about 400 ng/mL, including about 5 ng/mL, about 10 ng/mL, about 20 ng/mL, about 30 ng/mL, about 40 ng/mL, about 50 ng/mL, about 60 ng/mL, about 70 ng/mL, about 80 ng/mL, about 90 ng/mL, about 100 ng/mL, about 110 ng/mL, about 120 ng/mL, about 130 ng/mL, about 140 ng/mL, about 150 ng/mL, about 160 ng/mL, about 170 ng/mL about 180 ng/mL, about 190 ng/mL, about 200 ng/mL, about 210 ng/mL, about 220 ng/mL, about 230 ng/mL, about 240 ng/mL, about 250 ng/mL, about 260 ng/mL, about 270 ng/mL about 280 ng/mL, about 290 ng/mL, about 300 ng/mL, about 310 ng/mL, about 320 ng/mL, about 330 ng/mL, about 340 ng/mL, about 350 ng/mL, about 360 ng/mL, about 370 ng/mL about 380 ng/mL, about 390 ng/mL, and about 400 ng/mL, including all ranges there between. In other embodiments, the composition (b) of the present disclosure include compositions in which the steady state blood plasma Cmax of ketamine that falls within 80% to 125% of any of the steady state blood plasma Cmax values for ketamine provided herein.

In some embodiments, upon administration to a patient in need thereof the compositions of the present disclosure provide steady state plasma Cmax levels of norketamine (a ketamine and esketamine metabolite) that correlate to one or more statistically significant therapeutic effects. In some embodiments, the therapeutically effective steady state plasma Cmax levels of norketamine provided by the composition (b) of the present invention upon administration to a patient in need thereof range from about 5 ng/mL to about 400 ng/mL, including about 5 ng/mL, about 10 ng/mL, about 20 ng/mL, about 30 ng/mL, about 40 ng/mL, about 50 ng/mL, about 60 ng/mL, about 70 ng/mL, about 80 ng/mL, about 90 ng/mL, about 100 ng/mL, about 110 ng/mL, about 120 ng/mL, about 130 ng/mL, about 140 ng/mL, about 150 ng/mL, about 160 ng/mL, about 170 ng/mL about 180 ng/mL, about 190 ng/mL, about 200 ng/mL, about 210 ng/mL, about 220 ng/mL, about 230 ng/mL, about 240 ng/mL, about 250 ng/mL, about 260 ng/mL, about 270 ng/mL about 280 ng/mL, about 290 ng/mL, about 300 ng/mL, about 310 ng/mL, about 320 ng/mL, about 330 ng/mL, about 340 ng/mL, about 350 ng/mL, about 360 ng/mL, about 370 ng/mL about 380 ng/mL, about 390 ng/mL, and about 400 ng/mL, including all ranges there between. In other embodiments, the compositions of the present disclosure include composition (b) in which the steady state blood plasma Cmax of norketamine that falls within 80% to 125% of any of the steady state blood plasma Cmax values for norketamine provided herein.

In some embodiments, the present disclosure provides compositions and kits comprising a salt of Compound 1. In some embodiments, the salt of Compound 1 is Compound 1 Hydrobromide, Compound 1 Citrate, Compound 1 L-Malate, Compound 1 Mesylate, Compound 1 Phosphate, Compound 1 L(+)-Tartrate, Compound 1 Hydrochloride, Compound 1 Tosylate, Compound 1 Glucuronate, or Compound 1 Ethanesulfonate.

### Co-Therapy

While the compositions of the present disclosure can be administered as the only active pharmaceutical ingredients (e.g., GABA-A PAM and NMDA antagonist, NMDA Negative Allosteric Modulator or NMDA partial agonist) or the only active anti-depressant ingredients in the methods described herein, in other embodiments they can also be used in combination with one or more ingredients that are known to be therapeutically effective against depression and/or complement the antidepressant effect of the GABA-A PAM and/or the NMDA antagonist, NMDA Negative Allosteric Modulator, NMDA open channel blocker, or NMDA partial agonist ingredients.

For example, in some embodiments, the present methods can employ a GABA-A PAM and a NMDA antagonist, in conjunction with one or more additional anti-antidepressants agents.

In some embodiments, the GABA-A PAM and the NMDA antagonist, NMDA Negative Allosteric Modulator or NMDA partial agonist are administered in combination with an additional anti-depressant agent, e.g., co-formulated or administered separately. In some embodiments, the GABA-A PAM and the NMDA antagonist, NMDA Negative Allosteric Modulator or NMDA partial agonist are administered in conjunction with one or more selective serotonin reuptake inhibitors, serotonin norepinephrine reuptake inhibitors, tricyclic antidepressants, monoamine oxidase inhibitors, mirtazapine, bupropion, lamotrigine, atypical antipsychotics, or combinations thereof. In some embodiments, the GABA-A PAM and the NMDA antagonist, NMDA Negative Allosteric Modulator or NMDA partial agonist are administered in combination with electroconvulsive therapy (ECT). In some embodiments, the GABA-A PAM and the NMDA antagonist, NMDA Negative Allosteric Modulator or NMDA partial agonist are administered in combination with transcranial magnetic stimulation (TMS).

In some embodiments, the GABA-A PAM and the NMDA antagonist, NMDA Negative Allosteric Modulator or NMDA partial agonist are administered in conjunction with one or more selective serotonin reuptake inhibitors. In some embodiments, the one or more selective serotonin reuptake inhibitors is selected from the group consisting of fluoxetine, escitalopram, citalopram, sertraline, and paroxetine.

In some embodiments, the GABA-A PAM and the NMDA antagonist, NMDA Negative Allosteric Modulator or NMDA partial agonist are administered in conjunction with one or more serotonin norepinephrine reuptake inhibitors. In some embodiments, the one or more serotonin norepinephrine reuptake inhibitors is selected from the group consisting of venlafaxine and duloxetine.

In some embodiments, the GABA-A PAM and the NMDA antagonist, NMDA Negative Allosteric Modulator or NMDA partial agonist are administered in conjunction with one or more tricyclic antidepressants. In some embodiments, the one or more tricyclic antidepressants is selected from the group consisting of amitriptyline, imipramine, and nortriptyline.

In some embodiments, the GABA-A PAM and the NMDA antagonist, NMDA Negative Allosteric Modulator or NMDA partial agonist are administered in conjunction with one or monoamine oxidase inhibitors. In some embodiments, the one or more monoamine oxidase inhibitors is selected from the group consisting of phenelzine and tranylcypromine.

In some embodiments, the GABA-A PAM and the NMDA antagonist, NMDA Negative Allosteric Modulator or NMDA partial agonist are administered in conjunction with one or more atypical antipsychotics. In some embodiments, the one or more atypical antipsychotics is selected from the group consisting of lurasidone, aripiprazole, risperidone, olanzapine, quetiapine, ziprasidone, clozapine, iloperidone, paliperidone, asenapine and olanzapine/fluoxetine.

### Dosing

The invention provides methods for treating depression by administering an effective amount of a GABA-A PAM and an effective amount of an NMDA antagonists, NMDA Negative Allosteric Modulators or NMDA partial agonists to a patient in need thereof. An effective amount is an amount sufficient to eliminate or significantly reduce depression symptoms or to alleviate those symptoms (e.g., reduce the symptoms, such as depressed mood, compared to the symptoms present prior to treatment).

In some embodiments, the present disclosure provides methods for treating pain by administering an effective amount of a GABA-A PAM and an effective amount of an NMDA antagonists, NMDA Negative Allosteric Modulators or NMDA partial agonists to a patient in need thereof. An effective amount is an amount sufficient to eliminate or significantly reduce pain symptoms or to alleviate those symptoms (e.g., reduce the symptoms, such as acute pain, compared to the symptoms present prior to treatment).

In some embodiments, the present disclosure provides methods for treating adjustment disorder by administering an effective amount of a GABA-A PAM and an effective amount of an NMDA antagonists, NMDA Negative Allosteric Modulators or NMDA partial agonists to a patient in need thereof. An effective amount is an amount sufficient to eliminate or significantly reduce adjustment disorder symptoms or to alleviate those symptoms (e.g., reduce the symptoms, such as depressed mood and/or anxiety, compared to the symptoms present prior to treatment).

According to some embodiments of the present invention, administering a GABA-A PAM and an NMDA antagonist, NMDA Negative Allosteric Modulator or NMDA partial agonist provides statistically significant therapeutic effect. In one embodiment, the statistically significant therapeutic effect is determined based on one or more standards or criteria provided by one or more regulatory agencies in the United States, e.g., FDA or other countries (such as Australia). In another embodiment, the statistically significant therapeutic effect is determined based on results obtained from regulatory agency approved clinical trial set up and/or procedure.

In some embodiments, the statistically significant therapeutic effect is determined based on a patient population of at least about 20, 50, 60, 100, 200, 300, 400, 500, 600, 700, 800, 900, 1000 or 2000. In some embodiments, the statistically significant therapeutic effect is determined based on data obtained from randomized and double-blinded clinical trial set up. In some embodiments, the statistically significant therapeutic effect is determined based on data with a p value of less than or equal to about 0.05, 0.04, 0.03, 0.02 or 0.01. In some embodiments, the statistically significant therapeutic effect is determined based on data with a confidence interval greater than or equal to about 95%, 96%, 97%, 98% or 99%.

In some embodiments, the statistically significant therapeutic effect is determined by a randomized double-blind clinical trial of patients treated with a GABA-A PAM (for example, Compound 1) and an NMDA antagonist (for example, ketamine), NMDA Negative Allosteric Modulator or NMDA partial agonist and optionally in combination with standard care. In some embodiments, the statistically significant therapeutic effect is determined by a randomized clinical trial and using Hamilton Depression Rating Scale (HAM-D) as primary efficacy parameter and optionally in combination with any other commonly accepted criteria for depression assessment.

In general, statistical analysis can include any suitable method permitted by a regulatory agency, e.g., FDA in the US or Europe or any other country. In some embodiments, statistical analysis includes non-stratified analysis, log-rank analysis, e.g., from Kaplan-Meier, Jacobson-Truax, Gulliken-Lord-Novick, Edwards-Nunnally, Hageman-Arrindel and Hierarchical Linear Modeling (HLM) and Cox regression analysis.

Reduction of depression in patients with depressive conditions can be determined by various methods. In some embodiments, the effectiveness of a dosage regimen can be determined by evaluation via Hamilton Depression Rating Scale (HAM-D). In some embodiments, the effectiveness of a dosage regimen can be determined by evaluation via a Montgomery Asberg Depression Rating Scale (MADRS). In yet some other embodiments, the effectiveness of a dosage regimen can be determined by evaluation via HAM-D, MADRS, Hamilton Rating Scale for anxiety (HAM-A), Clinical Global Impression (CGI) subscale scores (i.e., Severity of Illness Subscale (CGI-S), Global Improvement Subscale (CGI-D, or Efficacy Index Subscale), Symptoms of Depression Questionnaire (SDQ), Pittsburgh Sleep Quality Index (PSQI), or any combination thereof.

In still another embodiment, the effectiveness of a dosage regimen can be determined by evaluation via a total HAM-D value as a primary efficacy endpoint in association with secondary efficacy endpoints such as the MADRS, HAM-A, CGI-S, CGI-I, SDQ, PSQI, or any combination thereof.

According to some embodiments of the present invention, the dosing frequency and dose amount per administration of the GABA-A PAM and the NMDA antagonist, NMDA Negative Allosteric Modulator or NMDA partial agonist are selected to provide therapeutic effects for the treatment of depression selected from major depressive disorder, major depressive disorder with suicidal risk, clinical depression, postnatal or postpartum depression, treatment resistant postpartum depression, perimenopausal depression, premenstrual dysphoric disorder (PMDD), atypical depression, melancholic depression, Psychotic Major Depression (PMD), catatonic depression, Seasonal Affective Disorder (SAD), persistent depressive disorder (dysthymia), double depression, Depressive Personality Disorder (DPD), Recurrent Brief Depression (RBD), minor depressive disorder, bipolar disorder or manic depressive disorder, bipolar depression with suicidal risk, post-traumatic stress disorders, depression caused by chronic medical conditions, depressive disorder due to another medical condition, treatment-resistant depression, refractory depression, substance/medication induced depressive disorder, depression with anxiety, suicidality, suicidal ideation, or suicidal behavior.

According to some embodiments of the present invention, the dosing frequency and dose amount per administration of the GABA-A PAM and the NMDA antagonist, NMDA Negative Allosteric Modulator or NMDA partial agonist are selected to provide therapeutic effects for the treatment of major depressive disorder. In some embodiments, the dosing frequency and amount per administration of the GABA-A PAM and the NMDA antagonist, NMDA Negative Allosteric Modulator or NMDA partial agonist are selected to provide for the treatment of moderate major depressive disorder. In some embodiments, the dosing frequency and amount per administration of the GABA-A PAM and the NMDA antagonist, NMDA Negative Allosteric Modulator or NMDA partial agonist are selected to provide for the treatment of severe major depressive disorder. In some embodiments, the dosing frequency and amount per administration of the GABA-A PAM and the NMDA antagonist, NMDA Negative Allosteric Modulator or NMDA partial agonist are selected to provide for the treatment of severe major depressive disorder in a patient having a total HAM-D value of at least 22.

According to some embodiments of the present invention, the dosing frequency and dose amount per administration of the GABA-A PAM and the NMDA antagonist, NMDA Negative Allosteric Modulator or NMDA partial agonist are selected to provide therapeutic effects for the treatment of depression that is refractory to other treatments (i.e., treatment resistant depression).

According to some embodiments of the present invention, the dosing frequency and dose amount per administration of the GABA-A PAM and the NMDA antagonist, NMDA Negative Allosteric Modulator or NMDA partial agonist are selected to provide therapeutic effects for the treatment of depression that is partially responsive to other antidepressant therapies. According to some embodiments of the present invention, the dosing frequency and dose amount per administration of the GABA-A PAM and the NMDA antagonist, NMDA Negative Allosteric Modulator or NMDA partial agonist are selected to provide an adjunctive treatment for major depression. According to some embodiments of the present invention, the dosing frequency and dose amount per administration of the GABA-A PAM and the NMDA antagonist, NMDA Negative Allosteric Modulator or NMDA partial agonist are selected to provide therapeutic effects for the treatment of depression that is partially responsive to treatment with SSRIs. According to some embodiments of the present invention, the dosing frequency and dose amount per administration of the GABA-A PAM and the NMDA antagonist, NMDA Negative Allosteric Modulator or NMDA partial agonist are selected to provide therapeutic effects for the treatment of depression that is partially responsive to treatment with NMDA antagonists, NMDA Negative Allosteric Modulators or NMDA partial agonists. In some embodiments, the dosing frequency and dose amount per administration of the GABA-A PAM and the NMDA antagonist, NMDA Negative Allosteric Modulator or NMDA partial agonist are selected to provide therapeutic effects for the treatment of depression that is partially responsive to treatment with ketamine.

In some embodiments, the dosing frequency and dose amount per administration of the GABA-A PAM and the NMDA antagonist, NMDA Negative Allosteric Modulator or NMDA partial agonist are selected to provide therapeutic effects for the treatment of MDD that is partially responsive to other antidepressant therapies. In some embodiments, the MDD patient that is partially responsive to other antidepressant therapies is an adult patient meeting DSM-IV criteria for MDD who had had an inadequate response to prior antidepressant therapy (1 to 3 courses) in the current episode and who had demonstrated an inadequate response to 8 weeks of prospective antidepressant therapy. Inadequate response for prospective treatment is defined as less than 50% improvement on the 17-item version of the Hamilton Depression Rating Scale (HAM-D), minimal HAM-D score of 14, and a Clinical Global Impressions Improvement rating of no better than minimal improvement. Inadequate response to prior treatment is defined as less than 50% improvement as perceived by the patient after a minimum of 6 weeks of antidepressant therapy at or above the minimal effective dose. In some embodiments, the dosing frequency and dose amount per administration of the GABA-A PAM and the NMDA antagonist, NMDA Negative Allosteric Modulator or NMDA partial agonist are selected to provide therapeutic effects for the treatment of moderate MDD that is partially responsive to other antidepressant therapies. In some embodiments, the dosing frequency and dose amount per administration of the GABA-A PAM and the NMDA antagonist, NMDA Negative Allosteric Modulator or NMDA partial agonist are selected to provide therapeutic effects for the treatment of severe MDD that is partially responsive to other antidepressant therapies. In some embodiments, the dosing frequency and dose amount per administration of the GABA-A PAM and the NMDA antagonist, NMDA Negative Allosteric Modulator or NMDA partial agonist are selected to provide therapeutic effects for the treatment of MDD that is partially responsive to treatment with SSRIs. In some embodiments, the dosing frequency and dose amount per administration of the GABA-A PAM and the NMDA antagonist, NMDA Negative Allosteric Modulator or NMDA partial agonist are selected to provide therapeutic effects for the treatment of moderate MDD that is partially responsive to treatment with SSRIs. In some embodiments, the dosing frequency and dose amount per administration of the GABA-A PAM and the NMDA antagonist, NMDA Negative Allosteric Modulator or NMDA partial agonist are selected to provide therapeutic effects for the treatment of severe MDD that is partially responsive to treatment with SSRIs.

In some embodiments, the dosing frequency and dose amount per administration of the GABA-A PAM and the NMDA antagonist, NMDA Negative Allosteric Modulator or NMDA partial agonist are selected to provide therapeutic effects for the treatment of depression and that do not substantially sedate the patient (i.e., the MDD is treated without substantially sedating the treated patient). In some embodiments, the dosing frequency and dose amount per administration of the GABA-A PAM and the NMDA antagonist, NMDA Negative Allosteric Modulator or NMDA partial agonist are selected to provide therapeutic effects for the treatment of MDD and that do not substantially sedate the patient. In some embodiments, the dosing frequency and dose amount per administration of the GABA-A PAM and the NMDA antagonist, NMDA Negative Allosteric Modulator or NMDA partial agonist are selected to provide therapeutic effects for the treatment of moderate MDD and that do not substantially sedate the patient. In some embodiments, the dosing frequency and dose amount per administration of the GABA-A PAM and the NMDA antagonist, NMDA Negative Allosteric Modulator or NMDA partial agonist are selected to provide therapeutic effects for the treatment of severe MDD and that do not substantially sedate the patient.

In some embodiments, the dosing frequency and dose amount per administration of the GABA-A PAM and the NMDA antagonist, NMDA Negative Allosteric Modulator or NMDA partial agonist are selected to provide therapeutic effects for the treatment of depression such that the therapeutically effective dose of GABA-A PAM and the NMDA antagonist, NMDA Negative Allosteric Modulator or NMDA partial agonist is about 25% less than the therapeutically effective dose of GABA-A PAM and the NMDA antagonist, NMDA Negative Allosteric Modulator or NMDA partial agonist when the GABA-A PAM and NMDA antagonist, NMDA Negative Allosteric Modulator or NMDA partial agonist are not co-administered (i.e., there is a synergistic effect such that the dose of the GABA-A PAM and the NMDA antagonist, NMDA Negative Allosteric Modulator or NMDA partial agonist may be reduced by 25% compared to the effective dose required for monotherapy to provide therapeutic effectiveness).

In some embodiments, the NMDA antagonist is ketamine and the dosing frequency and dose amount per administration of the GABA-A PAM and the ketamine are selected to provide therapeutic effects for the treatment of depression and to mitigate the anxiety and dissociative symptoms of ketamine administration (e.g., the MDD is treated without, or with a substantial reduction in, the anxiety and dissociative symptoms sometimes observed with ketamine administration). In some embodiments, the dosing frequency and dose amount per administration of the GABA-A PAM and the ketamine are selected to provide therapeutic effects for the treatment of MDD and to mitigate the anxiety and dissociative symptoms of ketamine. In some embodiments, the dosing frequency and dose amount per administration of the GABA-A PAM and the ketamine are selected to provide therapeutic effects for the treatment of moderate MDD and to mitigate the anxiety and dissociative symptoms of ketamine. In some embodiments, the dosing frequency and dose amount per administration of the GABA-A PAM and the ketamine are selected to provide therapeutic effects for the treatment of severe MDD and to mitigate the anxiety and dissociative symptoms of ketamine.

In some embodiments, the NMDA antagonist is ketamine and the dosing frequency and dose amount per administration of the GABA-A PAM and the ketamine are selected to prevent the treated patient from habituating to the antidepressant effects of ketamine.

In some embodiments, the dosing frequency and dose amount per administration of the GABA-A PAM and NMDA antagonist to provide therapeutic effects for the treatment of depression in the patient that was previously treated with an NMDA antagonist but did not tolerate said treatment, and wherein the dosing frequency and dose amount per administration of the GABA-A PAM and NMDA antagonist are selected such that the treated patient tolerates treatment with a GABA-A PAM and an NMDA antagonist.

A patient's sedation level may be measured using methods that are known to those skilled in the art. For example, sedation level may be measured using the Modified Observer's Assessment of Alertness/Sedation Scale (G. Schmidt, et al., Comparative Evaluation of the Datex-Ohmeda S/5 Entropy Module and the Bispectral Index® Monitor during Propofol-Remifentanil Anesthesia. Anesthesiology 2004; 101:1283-90) or the Stanford Sleepiness Scale (Quantification of Sleepiness: A New Approach. Psychophysiology Volume 10, Issue 4, pages 431-436, July 1973).

In some embodiments, the dosing frequency and dose amount per administration of the GABA-A PAM and the NMDA antagonist, NMDA Negative Allosteric Modulator or NMDA partial agonist are selected to provide therapeutic effects for the treatment of depression and a Modified Observer's Assessment of Alertness/Sedation Scale (MOAS/S) score of at least 4.0. In some embodiments, the dosing frequency and dose amount per administration of the GABA-A PAM and the NMDA antagonist, NMDA Negative Allosteric Modulator or NMDA partial agonist are selected to provide therapeutic effects for the treatment of depression and a MOAS/S score of 4. In some embodiments, the dosing frequency and dose amount per administration of the GABA-A PAM and the NMDA antagonist, NMDA Negative Allosteric Modulator or NMDA partial agonist are selected to provide therapeutic effects for the treatment of depression and a MOAS/S score of 5.

In some embodiments, the dosing frequency and dose amount per administration of the GABA-A PAM and the NMDA antagonist, NMDA Negative Allosteric Modulator or NMDA partial agonist are selected to provide therapeutic effects for the treatment of depression and a Stanford Sleepiness Scale Score of less than 3.0. In some embodiments, the dosing frequency and dose amount per administration of the GABA-A PAM and the NMDA antagonist, NMDA Negative Allosteric Modulator or NMDA partial agonist are selected to provide therapeutic effects for the treatment of depression and a Stanford Sleepiness Scale Score of 2. In some embodiments, the dosing frequency and dose amount per administration of the GABA-A PAM and the NMDA antagonist, NMDA Negative Allosteric Modulator or NMDA partial agonist are selected to provide therapeutic effects for the treatment of depression and a Stanford Sleepiness Scale Score of 1.

According to some embodiments of the present invention, the dosing frequency and dose amount per administration of the GABA-A PAM and the NMDA antagonist, NMDA Negative Allosteric Modulator or NMDA partial agonist are selected to provide therapeutic effects for the treatment of a mood or affective disorder selected from perimenopause, generalized anxiety disorder, panic disorder, social anxiety disorder, post-traumatic stress disorder, specific phobia, and selective mutism.

In some embodiments, the dosing frequency and dose amount per administration of the GABA-A PAM and the NMDA antagonist, NMDA Negative Allosteric Modulator or NMDA partial agonist are selected to provide therapeutic effects for the treatment of perimenopause. In some embodiments, the dosing frequency and dose amount per administration of the GABA-A PAM and the NMDA antagonist, NMDA Negative Allosteric Modulator or NMDA partial agonist are selected to provide therapeutic effects for the treatment of perimenopausal depression. Methods of diagnosing perimenopausal depression are known to those skilled in the art, such as set forth in Pauline M. Maki, et al. Guidelines for the Evaluation and Treatment of Perimenopausal Depression: Summary and Recommendations. Journal of Women's Health (DOI: 10.1089/jwh.2018.27099.mensocrec). In some embodiments, the dosing frequency and dose amount per administration of the GABA-A PAM and the NMDA antagonist, NMDA Negative Allosteric Modulator or NMDA partial agonist are selected to provide therapeutic effects for the treatment of perimenopause anxiety. In some embodiments, the dosing frequency and dose amount per administration of the GABA-A PAM and the NMDA antagonist, NMDA Negative Allosteric Modulator or NMDA partial agonist are selected to provide therapeutic effects for the treatment of perimenopause agitation.

In some embodiments, the dosing frequency and dose amount per administration of the GABA-A PAM and the NMDA antagonist, NMDA Negative Allosteric Modulator or NMDA partial agonist are selected to provide therapeutic effects for the treatment of menopause anxiety or postmenopause anxiety.

In some embodiments, the dosing frequency and dose amount per administration of the GABA-A PAM and the NMDA antagonist, NMDA Negative Allosteric Modulator or NMDA partial agonist are selected to provide therapeutic effects for the treatment of menopause agitation or postmenopause agitation.

In some embodiments, the dosing frequency and dose amount per administration of the GABA-A PAM and the NMDA antagonist, NMDA Negative Allosteric Modulator or NMDA partial agonist are selected to provide therapeutic effects for the acute treatment of depression. In some embodiments, the dosing frequency and dose amount per administration of the GABA-A PAM and the NMDA antagonist, NMDA Negative Allosteric Modulator or NMDA partial agonist are selected to provide therapeutic effects for the acute treatment of depression and, after the acute treatment of depression, the GABA-A PAM and the NMDA antagonist, NMDA Negative Allosteric Modulator or NMDA partial agonist is no longer administered and a dosing frequency and dose amount of second anti-depressant agent is selected to provide therapeutic effects for the chronic treatment of depression.

In some embodiments, the dosing frequency and dose amount per administration of the GABA-A PAM and the NMDA antagonist, NMDA Negative Allosteric Modulator or NMDA partial agonist are selected to provide therapeutic effects for the acute treatment of depression and, after the acute treatment of depression, a dosing frequency and dose amount of the GABA-A PAM and the NMDA antagonist, NMDA Negative Allosteric Modulator or NMDA partial agonist is selected to provide therapeutic effects for the chronic treatment of depression. In some embodiments, the daily dosing of the GABA-A PAM and the NMDA antagonist, NMDA Negative Allosteric Modulator or NMDA partial agonist for the acute treatment of depression is greater than the daily dosing of the GABA-A PAM and the NMDA antagonist, NMDA Negative Allosteric Modulator or NMDA partial agonist for the chronic treatment of depression.

In some embodiments, the dosing frequency and dose amount per administration of the GABA-A PAM and the NMDA antagonist, NMDA Negative Allosteric Modulator or NMDA partial agonist are selected to prevent the recurrence of depression. In some embodiments, the dosing frequency and dose amount per administration of the GABA-A PAM and the NMDA antagonist, NMDA Negative Allosteric Modulator or NMDA partial agonist are selected to maintain remission of depression.

In some embodiments, the GABA-A PAM is administered on a once a day or twice a day basis for at least a week, for example, about a week, about 2 weeks, about 3 weeks, about 4 weeks, about 5 weeks, about 6 weeks, about 7 weeks, about 8 weeks, about 9 weeks, about 10 weeks, about 12 weeks, about 18 weeks, about 24 weeks, and about 50 weeks.

In some embodiments, at least about 5 mg or about 5 mg of Compound 1 or a pharmaceutically acceptable salt thereof is administered on a once a day or twice a day basis for at least a week. In some embodiments, at least about 10 mg or about 10 mg of Compound 1 or a pharmaceutically acceptable salt thereof is administered on a once a day or twice a day basis for at least a week. In some embodiments, at least about 15 mg or about 15 mg of Compound 1 or a pharmaceutically acceptable salt thereof is administered on a once a day or twice a day basis for at least a week. In some embodiments, at least about 20 mg or about 20 mg of Compound 1 or a pharmaceutically acceptable salt thereof is administered on a once a day or twice a day basis for at least a week. In some embodiments, at least about 25 mg or about 25 mg of Compound 1 or a pharmaceutically acceptable salt thereof is administered on a once a day or twice a day basis for at least a week. In some embodiments, at least about 30 mg or about 30 mg of Compound 1 or a pharmaceutically acceptable salt thereof is administered on a once a day or twice a day basis for at least a week. In some embodiments, at least about 35 mg or about 35 mg of Compound 1 or a pharmaceutically acceptable salt thereof is administered on a once a day or twice a day basis for at least a week. In some embodiments, at least about 40 mg or about 40 mg of Compound 1 or a pharmaceutically acceptable salt thereof is administered on a once a day or twice a day basis for at least a week. In some embodiments, at least about 45 mg or about 45 mg of Compound 1 or a pharmaceutically acceptable salt thereof is administered on a once a day or twice a day basis for at least a week. In some embodiments, at least about 50 mg or about 50 mg of Compound 1 or a pharmaceutically acceptable salt thereof is administered on a once a day or twice a day basis for at least a week. In some embodiments, at least about 55 mg or about 55 mg of Compound 1 or a pharmaceutically acceptable salt thereof is administered on a once a day or twice a day basis for at least a week. In some embodiments, at least about 60 mg or about 60 mg of Compound 1 or a pharmaceutically acceptable salt thereof is administered on a once a day or twice a day basis for at least a week. In some embodiments, at least about 65 mg or about 65 mg of Compound 1 or a pharmaceutically acceptable salt thereof is administered on a once a day basis for at least a week. In some embodiments, at least about 70 mg or about 70 mg of Compound 1 or a pharmaceutically acceptable salt thereof is administered on a once a day basis for at least a week. In some embodiments, at least about 75 mg or about 75 mg of Compound 1 or a pharmaceutically acceptable salt thereof is administered on a once a day basis for at least a week. In some embodiments, at least about 80 mg or about 80 mg of Compound 1 or a pharmaceutically acceptable salt thereof is administered on a once a day basis for at least a week. In some embodiments, at least about 85 mg or about 85 mg of Compound 1 or a pharmaceutically acceptable salt thereof is administered on a once a day basis for at least a week. In some embodiments, at least about 90 mg or about 90 mg of Compound 1 or a pharmaceutically acceptable salt thereof is administered on a once a day basis for at least a week. In some embodiments, at least about 95 mg or about 95 mg of Compound 1 or a pharmaceutically acceptable salt thereof is administered on a once a day basis for at least a week. In some embodiments, at least about 100 mg or about 100 mg of Compound 1 or a pharmaceutically acceptable salt thereof is administered on a once a day basis for at least a week. In some embodiments, at least about 105 mg or about 105 mg of Compound 1 or a pharmaceutically acceptable salt thereof is administered on a once a day basis for at least a week. In some embodiments, at least about 110 mg or about 110 mg of Compound 1 or a pharmaceutically acceptable salt thereof is administered on a once a day basis for at least a week. In some embodiments, at least about 115 mg or about 115 mg of Compound 1 or a pharmaceutically acceptable salt thereof is administered on a once a day basis for at least a week. In some embodiments, at least about 120 mg or about 120 mg of Compound 1 or a pharmaceutically acceptable salt thereof is administered on a once a day basis for at least a week.

In some embodiments, the NMDA antagonist, NMDA Negative Allosteric Modulator or NMDA partial agonist is administered on a once a day or twice a day basis for at least a week, for example, about a week, about 2 weeks, about 3 weeks, about 4 weeks, about 5 weeks, about 6 weeks, about 7 weeks, about 8 weeks, about 9 weeks, about 10 weeks, about 12 weeks, about 18 weeks, about 24 weeks, and about 50 weeks. In some embodiments, the NMDA antagonist, NMDA Negative Allosteric Modulator or NMDA partial agonist is administered on a once a week, once every 2 week, once every 4 week basis for about 4 weeks, about 5 weeks, about 6 weeks, about 7 weeks, about 8 weeks, about 9 weeks, about 10 weeks, about 12 weeks, about 18 weeks, about 24 weeks, and about 50 weeks.

In some embodiments, the NMDA antagonist is ketamine or a pharmaceutically acceptable salt thereof. In some embodiments, at least about 1 mg or about 1 mg of ketamine or a pharmaceutically acceptable salt thereof is administered on a once a day or twice a day basis for at least a week. In some embodiments, at least about 5 mg or about 5 mg of ketamine or a pharmaceutically acceptable salt thereof is administered on a once a day or twice a day basis for at least a week. In some embodiments, at least about 10 mg or about 10 mg of ketamine or a pharmaceutically acceptable salt thereof is administered on a once a day or twice a day basis for at least a week. In some embodiments, at least about 15 mg or about 15 mg of ketamine or a pharmaceutically acceptable salt thereof is administered on a once a day or twice a day basis for at least a week. In some embodiments, at least about 20 mg or about 20 mg of ketamine or a pharmaceutically acceptable salt thereof is administered on a once a day or twice a day basis for at least a week. In some embodiments, at least about 25 mg or about 25 mg of ketamine or a pharmaceutically acceptable salt thereof is administered on a once a day or twice a day basis for at least a week. In some embodiments, at least about 30 mg or about 30 mg of ketamine or a pharmaceutically acceptable salt thereof is administered on a once a day or twice a day basis for at least a week. In some embodiments, at least about 35 mg or about 35 mg of ketamine or a pharmaceutically acceptable salt thereof is administered on a once a day or twice a day basis for at least a week. In some embodiments, at least about 40 mg or about 40 mg of ketamine or a pharmaceutically acceptable salt thereof is administered on a once a day or twice a day basis for at least a week. In some embodiments, at least about 45 mg or about 45 mg of ketamine or a pharmaceutically acceptable salt thereof is administered on a once a day or twice a day basis for at least a week. In some embodiments, at least about 50 mg or about 50 mg of ketamine or a pharmaceutically acceptable salt thereof is administered on a once a day or twice a day basis for at least a week. In some embodiments, at least about 55 mg or about 55 mg of ketamine or a pharmaceutically acceptable salt thereof is administered on a once a day or twice a day basis for at least a week. In some embodiments, at least about 60 mg or about 60 mg of ketamine or a pharmaceutically acceptable salt thereof is administered on a once a day or twice a day basis for at least a week. In some embodiments, at least about 65 mg or about 65 mg of ketamine or a pharmaceutically acceptable salt thereof is administered on a once a day basis for at least a week. In some embodiments, at least about 70 mg or about 70 mg of ketamine or a pharmaceutically acceptable salt thereof is administered on a once a day basis for at least a week. In some embodiments, at least about 75 mg or about 75 mg of ketamine or a pharmaceutically acceptable salt thereof is administered on a once a day basis for at least a week. In some embodiments, at least about 80 mg or about 80 mg of ketamine or a pharmaceutically acceptable salt thereof is administered on a once a day basis for at least a week. In some embodiments, at least about 85 mg or about 85 mg of ketamine or a pharmaceutically acceptable salt thereof is administered on a once a day basis for at least a week. In some embodiments, at least about 90 mg or about 90 mg of ketamine or a pharmaceutically acceptable salt thereof is administered on a once a day basis for at least a week. In some embodiments, at least about 95 mg or about 95 mg of ketamine or a pharmaceutically acceptable salt thereof is administered on a once a day basis for at least a week. In some embodiments, at least about 100 mg or about 100 mg of ketamine or a pharmaceutically acceptable salt thereof is administered on a once a day basis for at least a week. In some embodiments, at least about 105 mg or about 105 mg of ketamine or a pharmaceutically acceptable salt thereof is administered on a once a day basis for at least a week. In some embodiments, at least about 110 mg or about 110 mg of ketamine or a pharmaceutically acceptable salt thereof is administered on a once a day basis for at least a week. In some embodiments, at least about 115 mg or about 115 mg of ketamine or a pharmaceutically acceptable salt thereof is administered on a once a day basis for at least a week. In some embodiments, at least about 120 mg or about 120 mg of ketamine or a pharmaceutically acceptable salt thereof is administered on a once a day basis for at least a week. In some embodiments, at least about 125 mg or about 125 mg of ketamine or a pharmaceutically acceptable salt thereof is administered on a once a day or twice a day basis for at least a week. In some embodiments, at least about 130 mg or about 130 mg of ketamine or a pharmaceutically acceptable salt thereof is administered on a once a day or twice a day basis for at least a week. In some embodiments, at least about 135 mg or about 135 mg of ketamine or a pharmaceutically acceptable salt thereof is administered on a once a day or twice a day basis for at least a week. In some embodiments, at least about 140 mg or about 140 mg of ketamine or a pharmaceutically acceptable salt thereof is administered on a once a day or twice a day basis for at least a week. In some embodiments, at least about 145 mg or about 145 mg of ketamine or a pharmaceutically acceptable salt thereof is administered on a once a day or twice a day basis for at least a week. In some embodiments, at least about 150 mg or about 150 mg of ketamine or a pharmaceutically acceptable salt thereof is administered on a once a day or twice a day basis for at least a week. In some embodiments, at least about 155 mg or about 155 mg of ketamine or a pharmaceutically acceptable salt thereof is administered on a once a day or twice a day basis for at least a week. In some embodiments, at least about 160 mg or about 160 mg of ketamine or a pharmaceutically acceptable salt thereof is administered on a once a day or twice a day basis for at least a week. In some embodiments, at least about 165 mg or about 165 mg of ketamine or a pharmaceutically acceptable salt thereof is administered on a once a day basis for at least a week. In some embodiments, at least about 170 mg or about 170 mg of ketamine or a pharmaceutically acceptable salt thereof is administered on a once a day basis for at least a week. In some embodiments, at least about 175 mg or about 175 mg of ketamine or a pharmaceutically acceptable salt thereof is administered on a once a day basis for at least a week. In some embodiments, at least about 180 mg or about 180 mg of ketamine or a pharmaceutically acceptable salt thereof is administered on a once a day basis for at least a week. In some embodiments, at least about 185 mg or about 185 mg of ketamine or a pharmaceutically acceptable salt thereof is administered on a once a day basis for at least a week. In some embodiments, at least about 190 mg or about 190 mg of ketamine or a pharmaceutically acceptable salt thereof is administered on a once a day basis for at least a week. In some embodiments, at least about 195 mg or about 195 mg of ketamine or a pharmaceutically acceptable salt thereof is administered on a once a day basis for at least a week. In some embodiments, at least about 200 mg or about 200 mg of ketamine or a pharmaceutically acceptable salt thereof is administered on a once a day basis for at least a week. In some embodiments, at least about 205 mg or about 205 mg of ketamine or a pharmaceutically acceptable salt thereof is administered on a once a day basis for at least a week. In some embodiments, at least about 210 mg or about 210 mg of ketamine or a pharmaceutically acceptable salt thereof is administered on a once a day basis for at least a week. In some embodiments, at least about 215 mg or about 215 mg of ketamine or a pharmaceutically acceptable salt thereof is administered on a once a day basis for at least a week. In some embodiments, at least about 220 mg or about 220 mg of ketamine or a pharmaceutically acceptable salt thereof is administered on a once a day basis for at least a week. In some embodiments, at least about 225 mg or about 225 mg of ketamine or a pharmaceutically acceptable salt thereof is administered on a once a day or twice a day basis for at least a week. In some embodiments, at least about 230 mg or about 230 mg of ketamine or a pharmaceutically acceptable salt thereof is administered on a once a day or twice a day basis for at least a week. In some embodiments, at least about 235 mg or about 235 mg of ketamine or a pharmaceutically acceptable salt thereof is administered on a once a day or twice a day basis for at least a week. In some embodiments, at least about 240 mg or about 240 mg of ketamine or a pharmaceutically acceptable salt thereof is administered on a once a day or twice a day basis for at least a week. In some embodiments, at least about 245 mg or about 245 mg of ketamine or a pharmaceutically acceptable salt thereof is administered on a once a day or twice a day basis for at least a week. In some embodiments, at least about 250 mg or about 250 mg of ketamine or a pharmaceutically acceptable salt thereof is administered on a once a day or twice a day basis for at least a week. In some embodiments, the ketamine doses described herein are administered once a week or twice a week for at least one month. In some embodiments, the ketamine doses described herein are administered once a month or twice a month for at least one month.

According to the present invention, Compound 1 is administered on a once or twice a day basis to provide effective relief of the symptoms of depression (for example, major depressive disorder, major depressive disorder with suicidal risk, clinical depression, postnatal or postpartum depression, treatment resistant postpartum depression, perimenopausal depression, premenstrual dysphoric disorder (PMDD), atypical depression, melancholic depression, Psychotic Major Depression (PMD), catatonic depression, Seasonal Affective Disorder (SAD), persistent depressive disorder (dysthymia), double depression, Depressive Personality Disorder (DPD), Recurrent Brief Depression (RBD), minor depressive disorder, bipolar disorder or manic depressive disorder, bipolar depression with suicidal risk, post-traumatic stress disorders, depression caused by chronic medical conditions, depressive disorder due to another medical condition, treatment-resistant depression, refractory depression, substance/medication induced depressive disorder, depression with anxiety, suicidality, suicidal ideation, or suicidal behavior) or a mood or affective disorder selected from perimenopause, generalized anxiety disorder, panic disorder, social anxiety disorder, post-traumatic stress disorder, specific phobia, and selective mutism.

In some embodiments, the total daily dose of Compound 1 is about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, and about 120 mg.

In some embodiments, the total daily dose of Compound 1 is from about 5 mg to about 120 mg, including about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, and about 120 mg, including all ranges there between. In some embodiments, the total daily dose of Compound 1 is from about 15 mg to about 60 mg.

In some embodiments, ketamine is administered on a once or twice a day basis to provide effective relief of the symptoms of depression (for example, major depressive disorder, major depressive disorder with suicidal risk, clinical depression, postnatal or postpartum depression, treatment resistant postpartum depression, perimenopausal depression, premenstrual dysphoric disorder (PMDD), atypical depression, melancholic depression, Psychotic Major Depression (PMD), catatonic depression, Seasonal Affective Disorder (SAD), persistent depressive disorder (dysthymia), double depression, Depressive Personality Disorder (DPD), Recurrent Brief Depression (RBD), minor depressive disorder, bipolar disorder or manic depressive disorder, bipolar depression with suicidal risk, post-traumatic stress disorders, depression caused by chronic medical conditions, depressive disorder due to another medical condition, treatment-resistant depression, refractory depression, substance/medication induced depressive disorder, depression with anxiety, suicidality, suicidal ideation, or suicidal behavior) or a mood or affective disorder selected from perimenopause, generalized anxiety disorder, panic disorder, social anxiety disorder, post-traumatic stress disorder, specific phobia, and selective mutism.

In some embodiments, the total daily dose of ketamine is from about 1 mg to about 250 mg of ketamine or a pharmaceutically acceptable salt thereof, including about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, or about 250 mg, including all ranges there between.

In some embodiments, the total daily dose of ketamine is about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, or about 250 mg of ketamine or a pharmaceutically acceptable salt thereof.

In some embodiments, ketamine is administered on a once, twice or thrice a week basis to provide effective relief of the symptoms of depression or a mood or affective disorder selected from perimenopause, generalized anxiety disorder, panic disorder, social anxiety disorder, post-traumatic stress disorder, specific phobia, and selective mutism.

In some embodiments, the total weekly dose (or twice a week dose or three times a week) of ketamine is from about 1 mg to about 250 mg of ketamine or a pharmaceutically acceptable salt thereof, including about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, or about 250 mg, including all ranges there between.

In some embodiments, the total weekly dose (or twice a week dose or three times a week) of ketamine is about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, or about 250 mg of ketamine or a pharmaceutically acceptable salt thereof.

In the embodiments described herein, reference is made to the dose of Compound 1 and the dose of ketamine or esketamine for the treatment of depression (which includes acute treatment of depression and chronic treatment of depression). However, the present disclosure contemplates the disclosed doses for the treatment of a mood or affective disorder selected from perimenopause, generalized anxiety disorder, panic disorder, social anxiety disorder, post-traumatic stress disorder, specific phobia, and selective mutism as well as for the treatment of pain.

In some embodiments, the total daily dose of Compound 1 is at least about 5 mg a day for the treatment of depression. In some embodiments, the total daily dose of Compound 1 is at least about 10 mg a day for the treatment of depression. In some embodiments, the total daily dose of Compound 1 is at least about 15 mg a day for the treatment of depression. In some embodiments, the total daily dose of Compound 1 is at least about 20 mg a day for the treatment of depression. In some embodiments, the total daily dose of Compound 1 is at least about 25 mg a day for the treatment of depression. In some embodiments, the total daily dose of Compound 1 is at least about 30 mg a day for the treatment of depression. In some embodiments, the total daily dose of Compound 1 is at least about 35 mg a day for the treatment of depression. In some embodiments, the total daily dose of Compound 1 is at least about 40 mg a day for the treatment of depression. In some embodiments, the total daily dose of Compound 1 is at least about 45 mg a day for the treatment of depression. In some embodiments, the total daily dose of Compound 1 is at least about 50 mg a day for the treatment of depression. In some embodiments, the total daily dose of Compound 1 is at least about 55 mg a day for the treatment of depression. In some embodiments, the total daily dose of Compound 1 is at least about 60 mg a day for the treatment of depression. In some embodiments, the total daily dose of Compound 1 is at least about 65 mg a day for the treatment of depression. In some embodiments, the total daily dose of Compound 1 is at least about 70 mg a day for the treatment of depression. In some embodiments, the total daily dose of Compound 1 is at least about 75 mg a day for the treatment of depression. In some embodiments, the total daily dose of Compound 1 is at least about 80 mg a day for the treatment of depression. In some embodiments, the total daily dose of Compound 1 is at least about 85 mg a day for the treatment of depression. In some embodiments, the total daily dose of Compound 1 is at least about 90 mg a day for the treatment of depression. In some embodiments, the total daily dose of Compound 1 is at least about 95 mg a day for the treatment of depression. In some embodiments, the total daily dose of Compound 1 is at least about 100 mg a day for the treatment of depression. In some embodiments, the total daily dose of Compound 1 is at least about 105 mg a day for the treatment of depression. In some embodiments, the total daily dose of Compound 1 is at least about 110 mg a day for the treatment of depression. In some embodiments, the total daily dose of Compound 1 is at least about 115 mg a day for the treatment of depression. In some embodiments, the total daily dose of Compound 1 is at least about 120 mg a day for the treatment of depression.

In some embodiments, the total daily dose of Compound 1 is about 5 mg a day for the treatment of depression. In some embodiments, the total daily dose of Compound 1 is about 10 mg a day for the treatment of depression. In some embodiments, the total daily dose of Compound 1 is about 15 mg a day for the treatment of depression. In some embodiments, the total daily dose of Compound 1 is about 20 mg a day for the treatment of depression. In some embodiments, the total daily dose of Compound 1 is about 25 mg a day for the treatment of depression. In some embodiments, the total daily dose of Compound 1 is about 30 mg a day for the treatment of depression. In some embodiments, the total daily dose of Compound 1 is about 35 mg a day for the treatment of depression. In some embodiments, the total daily dose of Compound 1 is about 40 mg a day for the treatment of depression. In some embodiments, the total daily dose of Compound 1 is about 45 mg a day for the treatment of depression. In some embodiments, the total daily dose of Compound 1 is about 50 mg a day for the treatment of depression. In some embodiments, the total daily dose of Compound 1 is about 55 mg a day for the treatment of depression. In some embodiments, the total daily dose of Compound 1 is about 60 mg a day for the treatment of depression. In some embodiments, the total daily dose of Compound 1 is about 65 mg a day for the treatment of depression. In some embodiments, the total daily dose of Compound 1 is about 70 mg a day for the treatment of depression. In some embodiments, the total daily dose of Compound 1 is about 75 mg a day for the treatment of depression. In some embodiments, the total daily dose of Compound 1 is about 80 mg a day for the treatment of depression. In some embodiments, the total daily dose of Compound 1 is about 85 mg a day for the treatment of depression. In some embodiments, the total daily dose of Compound 1 is about 90 mg a day for the treatment of depression. In some embodiments, the total daily dose of Compound 1 is about 95 mg a day for the treatment of depression. In some embodiments, the total daily dose of Compound 1 is about 100 mg a day for the treatment of depression. In some embodiments, the total daily dose of Compound 1 is about 105 mg a day for the treatment of depression. In some embodiments, the total daily dose of Compound 1 is about 110 mg a day for the treatment of depression. In some embodiments, the total daily dose of Compound 1 is about 115 mg a day for the treatment of depression. In some embodiments, the total daily dose of Compound 1 is about 120 mg a day for the treatment of depression.

In some embodiments, about 5 mg of Compound 1 once a day is selected to provide a substantial reduction in depression. In some embodiments, about 5 mg of Compound 1 twice a day is selected to provide a substantial reduction in depression. In some embodiments, about 10 mg of Compound 1 once a day is selected to provide a substantial reduction in depression. In some embodiments, about 10 mg of Compound 1 twice a day is selected to provide a substantial reduction in depression. In some embodiments, about 15 mg of Compound 1 once a day is selected to provide a substantial reduction in depression. In some embodiments, about 15 mg of Compound 1 twice a day is selected to provide a substantial reduction in depression. In some embodiments, about 20 mg of Compound 1 once a day is selected to provide a substantial reduction in depression. In some embodiments, about 20 mg of Compound 1 twice a day is selected to provide a substantial reduction in depression. In some embodiments, about 25 mg of Compound 1 once a day is selected to provide a substantial reduction in depression. In some embodiments, about 25 mg of Compound 1 twice a day is selected to provide a substantial reduction in depression. In some embodiments, about 30 mg of Compound 1 once a day is selected to provide a substantial reduction in depression. In some embodiments, about 30 mg of Compound 1 twice a day is selected to provide a substantial reduction in depression. In some embodiments, about 30 mg of Compound 1 once a day is selected to provide a substantial reduction in depression. In some embodiments, about 30 mg of Compound 1 twice a day is selected to provide a substantial reduction in depression. In some embodiments, about 35 mg of Compound 1 once a day is selected to provide a substantial reduction in depression. In some embodiments, about 35 mg of Compound 1 twice a day is selected to provide a substantial reduction in depression. In some embodiments, about 40 mg of Compound 1 once a day is selected to provide a substantial reduction in depression. In some embodiments, about 40 mg of Compound 1 twice a day is selected to provide a substantial reduction in depression. In some embodiments, about 45 mg of Compound 1 once a day is selected to provide a substantial reduction in depression. In some embodiments, about 45 mg of Compound 1 twice a day is selected to provide a substantial reduction in depression. In some embodiments, about 50 mg of Compound 1 once a day is selected to provide a substantial reduction in depression. In some embodiments, about 50 mg of Compound 1 twice a day is selected to provide a substantial reduction in depression. In some embodiments, about 55 mg of Compound 1 once a day is selected to provide a substantial reduction in depression. In some embodiments, about 55 mg of Compound 1 twice a day is selected to provide a substantial reduction in depression. In some embodiments, about 60 mg of Compound 1 once a day is selected to provide a substantial reduction in depression. In some embodiments, about 60 mg of Compound 1 twice a day is selected to provide a substantial reduction in depression. In some embodiments, about 65 mg of Compound 1 once a day is selected to provide a substantial reduction in depression. In some embodiments, about 70 mg of Compound 1 once a day is selected to provide a substantial reduction in depression. In some embodiments, about 75 mg of Compound 1 once a day is selected to provide a substantial reduction in depression. In some embodiments, about 80 mg of Compound 1 once a day is selected to provide a substantial reduction in depression. In some embodiments, about 85 mg of Compound 1 once a day is selected to provide a substantial reduction in depression. In some embodiments, about 90 mg of Compound 1 once a day is selected to provide a substantial reduction in depression. In some embodiments, about 95 mg of Compound 1 once a day is selected to provide a substantial reduction in depression. In some embodiments, about 100 mg of Compound 1 once a day is selected to provide a substantial reduction in depression. In some embodiments, about 105 mg of Compound 1 once a day is selected to provide a substantial reduction in depression. In some embodiments, about 110 mg of Compound 1 once a day is selected to provide a substantial reduction in depression. In some embodiments, about 115 mg of Compound 1 once a day is selected to provide a substantial reduction in depression. In some embodiments, about 120 mg of Compound 1 once a day is selected to provide a substantial reduction in depression.

In the following embodiments, the total daily dose of ketamine for the treatment of depression is provided. However, the present disclosure contemplates embodiments where the described total daily doses are administered as total weekly doses, total biweekly doses and total monthly doses.

In some embodiments, the total daily dose of ketamine is at least about 1 mg a day for the treatment of depression. In some embodiments, the total daily dose of ketamine is at least about 5 mg a day for the treatment of depression. In some embodiments, the total daily dose of ketamine is at least about 10 mg a day for the treatment of depression. In some embodiments, the total daily dose of ketamine is at least about 15 mg a day for the treatment of depression. In some embodiments, the total daily dose of ketamine is at least about 20 mg a day for the treatment of depression. In some embodiments, the total daily dose of ketamine is at least about 25 mg a day for the treatment of depression. In some embodiments, the total daily dose of ketamine is at least about 30 mg a day for the treatment of depression. In some embodiments, the total daily dose of ketamine is at least about 35 mg a day for the treatment of depression. In some embodiments, the total daily dose of ketamine is at least about 40 mg a day for the treatment of depression. In some embodiments, the total daily dose of ketamine is at least about 45 mg a day for the treatment of depression. In some embodiments, the total daily dose of ketamine is at least about 50 mg a day for the treatment of depression. In some embodiments, the total daily dose of ketamine is at least about 55 mg a day for the treatment of depression. In some embodiments, the total daily dose of ketamine is at least about 60 mg a day for the treatment of depression. In some embodiments, the total daily dose of ketamine is at least about 65 mg a day for the treatment of depression. In some embodiments, the total daily dose of ketamine is at least about 70 mg a day for the treatment of depression. In some embodiments, the total daily dose of ketamine is at least about 75 mg a day for the treatment of depression. In some embodiments, the total daily dose of ketamine is at least about 80 mg a day for the treatment of depression. In some embodiments, the total daily dose of ketamine is at least about 85 mg a day for the treatment of depression. In some embodiments, the total daily dose of ketamine is at least about 90 mg a day for the treatment of depression. In some embodiments, the total daily dose of ketamine is at least about 95 mg a day for the treatment of depression. In some embodiments, the total daily dose of ketamine is at least about 100 mg a day for the treatment of depression. In some embodiments, the total daily dose of ketamine is at least about 105 mg a day for the treatment of depression. In some embodiments, the total daily dose of ketamine is at least about 110 mg a day for the treatment of depression. In some embodiments, the total daily dose of ketamine is at least about 115 mg a day for the treatment of depression. In some embodiments, the total daily dose of ketamine is at least about 120 mg a day for the treatment of depression. In some embodiments, the total daily dose of ketamine is at least about 125 mg a day for the treatment of depression. In some embodiments, the total daily dose of ketamine is at least about 130 mg a day for the treatment of depression. In some embodiments, the total daily dose of ketamine is at least about 135 mg a day for the treatment of depression. In some embodiments, the total daily dose of ketamine is at least about 140 mg a day for the treatment of depression. In some embodiments, the total daily dose of ketamine is at least about 145 mg a day for the treatment of depression. In some embodiments, the total daily dose of ketamine is at least about 150 mg a day for the treatment of depression. In some embodiments, the total daily dose of ketamine is at least about 155 mg a day for the treatment of depression. In some embodiments, the total daily dose of ketamine is at least about 160 mg a day for the treatment of depression. In some embodiments, the total daily dose of ketamine is at least about 165 mg a day for the treatment of depression. In some embodiments, the total daily dose of ketamine is at least about 170 mg a day for the treatment of depression. In some embodiments, the total daily dose of ketamine is at least about 175 mg a day for the treatment of depression. In some embodiments, the total daily dose of ketamine is at least about 180 mg a day for the treatment of depression. In some embodiments, the total daily dose of ketamine is at least about 185 mg a day for the treatment of depression. In some embodiments, the total daily dose of ketamine is at least about 190 mg a day for the treatment of depression. In some embodiments, the total daily dose of ketamine is at least about 195 mg a day for the treatment of depression. In some embodiments, the total daily dose of ketamine is at least about 200 mg a day for the treatment of depression. In some embodiments, the total daily dose of ketamine is at least about 205 mg a day for the treatment of depression. In some embodiments, the total daily dose of ketamine is at least about 210 mg a day for the treatment of depression. In some embodiments, the total daily dose of ketamine is at least about 215 mg a day for the treatment of depression. In some embodiments, the total daily dose of ketamine is at least about 220 mg a day for the treatment of depression. In some embodiments, the total daily dose of ketamine is at least about 225 mg a day for the treatment of depression. In some embodiments, the total daily dose of ketamine is at least about 230 mg a day for the treatment of depression. In some embodiments, the total daily dose of ketamine is at least about 235 mg a day for the treatment of depression. In some embodiments, the total daily dose of ketamine is at least about 240 mg a day for the treatment of depression. In some embodiments, the total daily dose of ketamine is at least about 245 mg a day for the treatment of depression. In some embodiments, the total daily dose of ketamine is at least about 250 mg a day for the treatment of depression.

In some embodiments, the total daily dose of ketamine is about 5 mg a day for the treatment of depression. In some embodiments, the total daily dose of ketamine is about 10 mg a day for the treatment of depression. In some embodiments, the total daily dose of ketamine is about 15 mg a day for the treatment of depression. In some embodiments, the total daily dose of ketamine is about 20 mg a day for the treatment of depression. In some embodiments, the total daily dose of ketamine is about 25 mg a day for the treatment of depression. In some embodiments, the total daily dose of ketamine is about 30 mg a day for the treatment of depression. In some embodiments, the total daily dose of ketamine is about 35 mg a day for the treatment of depression. In some embodiments, the total daily dose of ketamine is about 40 mg a day for the treatment of depression. In some embodiments, the total daily dose of ketamine is about 45 mg a day for the treatment of depression. In some embodiments, the total daily dose of ketamine is about 50 mg a day for the treatment of depression. In some embodiments, the total daily dose of ketamine is about 55 mg a day for the treatment of depression. In some embodiments, the total daily dose of ketamine is about 60 mg a day for the treatment of depression. In some embodiments, the total daily dose of ketamine is about 65 mg a day for the treatment of depression. In some embodiments, the total daily dose of ketamine is about 70 mg a day for the treatment of depression. In some embodiments, the total daily dose of ketamine is about 75 mg a day for the treatment of depression. In some embodiments, the total daily dose of ketamine is about 80 mg a day for the treatment of depression. In some embodiments, the total daily dose of ketamine is about 85 mg a day for the treatment of depression. In some embodiments, the total daily dose of ketamine is about 90 mg a day for the treatment of depression. In some embodiments, the total daily dose of ketamine is about 95 mg a day for the treatment of depression. In some embodiments, the total daily dose of ketamine is about 100 mg a day for the treatment of depression. In some embodiments, the total daily dose of ketamine is about 105 mg a day for the treatment of depression. In some embodiments, the total daily dose of ketamine is about 110 mg a day for the treatment of depression. In some embodiments, the total daily dose of ketamine is about 115 mg a day for the treatment of depression. In some embodiments, the total daily dose of ketamine is about 120 mg a day for the treatment of depression. In some embodiments, the total daily dose of ketamine is about 125 mg a day for the treatment of depression. In some embodiments, the total daily dose of ketamine is about 130 mg a day for the treatment of depression. In some embodiments, the total daily dose of ketamine is about 135 mg a day for the treatment of depression. In some embodiments, the total daily dose of ketamine is about 140 mg a day for the treatment of depression. In some embodiments, the total daily dose of ketamine is about 145 mg a day for the treatment of depression. In some embodiments, the total daily dose of ketamine is about 150 mg a day for the treatment of depression. In some embodiments, the total daily dose of ketamine is about 155 mg a day for the treatment of depression. In some embodiments, the total daily dose of ketamine is about 160 mg a day for the treatment of depression. In some embodiments, the total daily dose of ketamine is about 165 mg a day for the treatment of depression. In some embodiments, the total daily dose of ketamine is about 170 mg a day for the treatment of depression. In some embodiments, the total daily dose of ketamine is about 175 mg a day for the treatment of depression. In some embodiments, the total daily dose of ketamine is about 180 mg a day for the treatment of depression. In some embodiments, the total daily dose of ketamine is about 185 mg a day for the treatment of depression. In some embodiments, the total daily dose of ketamine is about 190 mg a day for the treatment of depression. In some embodiments, the total daily dose of ketamine is about 195 mg a day for the treatment of depression. In some embodiments, the total daily dose of ketamine is about 200 mg a day for the treatment of depression. In some embodiments, the total daily dose of ketamine is about 205 mg a day for the treatment of depression. In some embodiments, the total daily dose of ketamine is about 210 mg a day for the treatment of depression. In some embodiments, the total daily dose of ketamine is about 215 mg a day for the treatment of depression. In some embodiments, the total daily dose of ketamine is about 220 mg a day for the treatment of depression. In some embodiments, the total daily dose of ketamine is about 225 mg a day for the treatment of depression. In some embodiments, the total daily dose of ketamine is about 230 mg a day for the treatment of depression. In some embodiments, the total daily dose of ketamine is about 235 mg a day for the treatment of depression. In some embodiments, the total daily dose of ketamine is about 240 mg a day for the treatment of depression. In some embodiments, the total daily dose of ketamine is about 245 mg a day for the treatment of depression. In some embodiments, the total daily dose of ketamine is about 250 mg a day for the treatment of depression.

In some embodiments, about 5 mg of ketamine once a day is selected to provide a substantial reduction in depression. In some embodiments, about 5 mg of ketamine twice a day is selected to provide a substantial reduction in depression. In some embodiments, about 10 mg of ketamine once a day is selected to provide a substantial reduction in depression. In some embodiments, about 10 mg of ketamine twice a day is selected to provide a substantial reduction in depression. In some embodiments, about 15 mg of ketamine once a day is selected to provide a substantial reduction in depression. In some embodiments, about 15 mg of ketamine twice a day is selected to provide a substantial reduction in depression. In some embodiments, about 20 mg of ketamine once a day is selected to provide a substantial reduction in depression. In some embodiments, about 20 mg of ketamine twice a day is selected to provide a substantial reduction in depression. In some embodiments, about 25 mg of ketamine once a day is selected to provide a substantial reduction in depression. In some embodiments, about 25 mg of ketamine twice a day is selected to provide a substantial reduction in depression. In some embodiments, about 30 mg of ketamine once a day is selected to provide a substantial reduction in depression. In some embodiments, about 30 mg of ketamine twice a day is selected to provide a substantial reduction in depression. In some embodiments, about 30 mg of ketamine once a day is selected to provide a substantial reduction in depression. In some embodiments, about 30 mg of ketamine twice a day is selected to provide a substantial reduction in depression. In some embodiments, about 35 mg of ketamine once a day is selected to provide a substantial reduction in depression. In some embodiments, about 35 mg of ketamine twice a day is selected to provide a substantial reduction in depression. In some embodiments, about 40 mg of ketamine once a day is selected to provide a substantial reduction in depression. In some embodiments, about 40 mg of ketamine twice a day is selected to provide a substantial reduction in depression. In some embodiments, about 45 mg of ketamine once a day is selected to provide a substantial reduction in depression. In some embodiments, about 45 mg of ketamine twice a day is selected to provide a substantial reduction in depression. In some embodiments, about 50 mg of ketamine once a day is selected to provide a substantial reduction in depression. In some embodiments, about 50 mg of ketamine twice a day is selected to provide a substantial reduction in depression. In some embodiments, about 55 mg of ketamine once a day is selected to provide a substantial reduction in depression. In some embodiments, about 55 mg of ketamine twice a day is selected to provide a substantial reduction in depression. In some embodiments, about 60 mg of ketamine once a day is selected to provide a substantial reduction in depression. In some embodiments, about 60 mg of ketamine twice a day is selected to provide a substantial reduction in depression. In some embodiments, about 65 mg of ketamine once a day is selected to provide a substantial reduction in depression. In some embodiments, about 70 mg of ketamine once a day is selected to provide a substantial reduction in depression. In some embodiments, about 75 mg of ketamine once a day is selected to provide a substantial reduction in depression. In some embodiments, about 80 mg of ketamine once a day is selected to provide a substantial reduction in depression. In some embodiments, about 85 mg of ketamine once a day is selected to provide a substantial reduction in depression. In some embodiments, about 90 mg of ketamine once a day is selected to provide a substantial reduction in depression. In some embodiments, about 95 mg of ketamine once a day is selected to provide a substantial reduction in depression. In some embodiments, about 100 mg of ketamine once a day is selected to provide a substantial reduction in depression. In some embodiments, about 105 mg of ketamine once a day is selected to provide a substantial reduction in depression. In some embodiments, about 110 mg of ketamine once a day is selected to provide a substantial reduction in depression. In some embodiments, about 115 mg of ketamine once a day is selected to provide a substantial reduction in depression. In some embodiments, about 120 mg of ketamine once a day is selected to provide a substantial reduction in depression. In some embodiments, about 125 mg of ketamine once a day is selected to provide a substantial reduction in depression. In some embodiments, about 125 mg of ketamine twice a day is selected to provide a substantial reduction in depression. In some embodiments, about 130 mg of ketamine once a day is selected to provide a substantial reduction in depression. In some embodiments, about 130 mg of ketamine twice a day is selected to provide a substantial reduction in depression. In some embodiments, about 135 mg of ketamine once a day is selected to provide a substantial reduction in depression. In some embodiments, about 135 mg of ketamine twice a day is selected to provide a substantial reduction in depression. In some embodiments, about 140 mg of ketamine once a day is selected to provide a substantial reduction in depression. In some embodiments, about 140 mg of ketamine twice a day is selected to provide a substantial reduction in depression. In some embodiments, about 145 mg of ketamine once a day is selected to provide a substantial reduction in depression. In some embodiments, about 145 mg of ketamine twice a day is selected to provide a substantial reduction in depression. In some embodiments, about 150 mg of ketamine once a day is selected to provide a substantial reduction in depression. In some embodiments, about 150 mg of ketamine twice a day is selected to provide a substantial reduction in depression. In some embodiments, about 155 mg of ketamine once a day is selected to provide a substantial reduction in depression. In some embodiments, about 155 mg of ketamine twice a day is selected to provide a substantial reduction in depression. In some embodiments, about 160 mg of ketamine once a day is selected to provide a substantial reduction in depression. In some embodiments, about 160 mg of ketamine twice a day is selected to provide a substantial reduction in depression. In some embodiments, about 165 mg of ketamine once a day is selected to provide a substantial reduction in depression. In some embodiments, about 165 mg of ketamine twice a day is selected to provide a substantial reduction in depression. In some embodiments, about 170 mg of ketamine once a day is selected to provide a substantial reduction in depression. In some embodiments, about 170 mg of ketamine twice a day is selected to provide a substantial reduction in depression. In some embodiments, about 175 mg of ketamine once a day is selected to provide a substantial reduction in depression. In some embodiments, about 175 mg of ketamine twice a day is selected to provide a substantial reduction in depression. In some embodiments, about 180 mg of ketamine once a day is selected to provide a substantial reduction in depression. In some embodiments, about 180 mg of ketamine twice a day is selected to provide a substantial reduction in depression. In some embodiments, about 185 mg of ketamine once a day is selected to provide a substantial reduction in depression. In some embodiments, about 185 mg of ketamine twice a day is selected to provide a substantial reduction in depression. In some embodiments, about 190 mg of ketamine once a day is selected to provide a substantial reduction in depression. In some embodiments, about 190 mg of ketamine twice a day is selected to provide a substantial reduction in depression. In some embodiments, about 195 mg of ketamine once a day is selected to provide a substantial reduction in depression. In some embodiments, about 195 mg of ketamine twice a day is selected to provide a substantial reduction in depression. In some embodiments, about 200 mg of ketamine once a day is selected to provide a substantial reduction in depression. In some embodiments, about 200 mg of ketamine twice a day is selected to provide a substantial reduction in depression. In some embodiments, about 205 mg of ketamine once a day is selected to provide a substantial reduction in depression. In some embodiments, about 205 mg of ketamine twice a day is selected to provide a substantial reduction in depression. In some embodiments, about 210 mg of ketamine once a day is selected to provide a substantial reduction in depression. In some embodiments, about 210 mg of ketamine twice a day is selected to provide a substantial reduction in depression. In some embodiments, about 215 mg of ketamine once a day is selected to provide a substantial reduction in depression. In some embodiments, about 215 mg of ketamine twice a day is selected to provide a substantial reduction in depression. In some embodiments, about 220 mg of ketamine once a day is selected to provide a substantial reduction in depression. In some embodiments, about 220 mg of ketamine twice a day is selected to provide a substantial reduction in depression. In some embodiments, about 225 mg of ketamine once a day is selected to provide a substantial reduction in depression. In some embodiments, about 225 mg of ketamine twice a day is selected to provide a substantial reduction in depression. In some embodiments, about 230 mg of ketamine once a day is selected to provide a substantial reduction in depression. In some embodiments, about 230 mg of ketamine twice a day is selected to provide a substantial reduction in depression. In some embodiments, about 230 mg of ketamine once a day is selected to provide a substantial reduction in depression. In some embodiments, about 230 mg of ketamine twice a day is selected to provide a substantial reduction in depression. In some embodiments, about 235 mg of ketamine once a day is selected to provide a substantial reduction in depression. In some embodiments, about 235 mg of ketamine twice a day is selected to provide a substantial reduction in depression. In some embodiments, about 240 mg of ketamine once a day is selected to provide a substantial reduction in depression. In some embodiments, about 240 mg of ketamine twice a day is selected to provide a substantial reduction in depression. In some embodiments, about 245 mg of ketamine once a day is selected to provide a substantial reduction in depression. In some embodiments, about 45 mg of ketamine twice a day is selected to provide a substantial reduction in depression. In some embodiments, about 250 mg of ketamine once a day is selected to provide a substantial reduction in depression. In some embodiments, about 250 mg of ketamine twice a day is selected to provide a substantial reduction in depression.

In some embodiments, the GABA-A PAM is Compound 1 or a pharmaceutically acceptable salt thereof and the NMDA antagonist is esketamine or a pharmaceutically acceptable salt therefore. In some embodiments, esketamine is administered on a once a week basis, on a twice a week basis or on an every other week basis to provide effective relief of the symptoms of depression (for example, major depressive disorder, major depressive disorder with suicidal risk, clinical depression, postnatal or postpartum depression, treatment resistant postpartum depression, perimenopausal depression, premenstrual dysphoric disorder (PMDD), atypical depression, melancholic depression, Psychotic Major Depression (PMD), catatonic depression, Seasonal Affective Disorder (SAD), persistent depressive disorder (dysthymia), double depression, Depressive Personality Disorder (DPD), Recurrent Brief Depression (RBD), minor depressive disorder, bipolar disorder or manic depressive disorder, bipolar depression with suicidal risk, post-traumatic stress disorders, depression caused by chronic medical conditions, depressive disorder due to another medical condition, treatment-resistant depression, refractory depression, substance/medication induced depressive disorder, depression with anxiety, suicidality, suicidal ideation, or suicidal behavior) or a mood or affective disorder selected from perimenopause, generalized anxiety disorder, panic disorder, social anxiety disorder, post-traumatic stress disorder, specific phobia, and selective mutism.

In some embodiments, the total weekly dose of esketamine administered intranasally is at least about 100 mg for the treatment of depression. In some embodiments, the total weekly dose of esketamine administered intranasally is at least about 160 mg for the treatment of depression. In some embodiments, the total weekly dose of esketamine administered intranasally is at least about 100 mg for the treatment of depression. In some embodiments, the total weekly dose of esketamine administered intranasally is at least about 160 mg for the treatment of depression. In some embodiments, the total biweekly (i.e., dosed every other week) dose of esketamine administered intranasally is at least about 50 mg for the treatment of depression. In some embodiments, the total biweekly (i.e., dosed every other week) dose of esketamine administered intranasally is at least about 80 mg for the treatment of depression.

In some embodiments, the dose of esketamine administered intranasally is about 56 mg once a week for the treatment of depression. In some embodiments, the dose of esketamine administered intranasally is about 84 mg once a week for the treatment of depression. In some embodiments, the dose of esketamine administered intranasally is about 56 mg twice a week for the treatment of depression. In some embodiments, the dose of esketamine administered intranasally is about 84 mg twice a week for the treatment of depression. In some embodiments, the dose of esketamine administered intranasally is about 56 mg once every other week for the treatment of depression. In some embodiments, the dose of esketamine administered intranasally is about 84 mg once every other week for the treatment of depression.

In some embodiments, about 56 mg of esketamine administered intranasally once a week is selected to provide a substantial reduction in depression. In some embodiments, about 84 mg of esketamine administered intranasally once a week is selected to provide a substantial reduction in depression. In some embodiments, about 56 mg of esketamine administered intranasally twice a week is selected to provide a substantial reduction in depression. In some embodiments, about 84 mg of esketamine administered intranasally twice a week is selected to provide a substantial reduction in depression. In some embodiments, about 56 mg of esketamine administered intranasally once every other week is selected to provide a substantial reduction in depression. In some embodiments, about 84 mg of esketamine administered intranasally once every other week is selected to provide a substantial reduction in depression.

According to some embodiments, the substantial reduction in depression provided by the methods of the present disclosure requires treatment for a specified time interval (e.g., at least one week) before the patient experiences substantial reduction of depression (i.e., there is an induction period before the patient experiences a substantial reduction in depression). In some embodiments, after treatment for at least one week, at least two weeks, at least three weeks, at least four weeks, at least five weeks, at least six weeks, at least seven weeks or at least eight weeks, the patient experiences a substantial reduction of depression compared to prior to the treatment. In some embodiments, after treatment for at least one week the patient experiences a substantial reduction of depression compared to prior to the treatment. According to this embodiment, the substantial reduction in depression may be expressed using any of the methods described herein (for example, decline in total Hamilton Depression Rating Scale (HAM-D) value compared to prior to the treatment, improvement in the Montgomery Asberg Depression Rating Scale value compared to prior to the treatment, etc.).

According to some embodiments, the substantial reduction in depression provided by the methods of the present disclosure is experienced by the patient after the first treatment (i.e., there is no induction period before the patient experiences a substantial reduction in depression). According to this embodiment, the substantial reduction in depression may be expressed using any of the methods described herein (for example, decline in total Hamilton Depression Rating Scale (HAM-D) value compared to prior to the treatment, improvement in the Montgomery Asberg Depression Rating Scale value compared to prior to the treatment, etc.).

The HAM-D is a depression rating scale consisting of 17 items, eight items are scored on a 5-point scale (ranging from 0 to 4), and 9 items are scores on a 3-point scale (ranging from 0 to 2). The total score of the 17 items ranges from 0 to 50 with higher scores indicating greater depression. The total score the 17 items is used to categorize the severity of depression: normal (total score between 0 and 7), mild depression (total score between 8 and 13), moderate depression (total score between 14-18), severe depression (total score between 19-22). Therefore, a decrease in the total score or on individual item scores indicates improvement Hamilton, M. A Rating Scale for Depression, Journal of Neurology, Neurosurgery, and Psychiatry. (1960) 23, pages 56-62.

In some embodiments, after the treatment the patient experiences a substantial reduction of depression that is characterized by at least about a 30% decline in total Hamilton Depression Rating Scale (HAM-D) value compared to prior to the treatment. In some embodiments, the reduction of depression is characterized by a decline in HAM-D value ranging from about 30% to about 100%, for example, about 30%, about 40%, about 50%, about 60%, about 70%, about 80%, about 90%, and about 100%, compared to prior to the treatment.

In some embodiments, after the treatment the patient experiences a substantial reduction of depression that is characterized by at least a one point decline in HAM-D value compared to prior to the treatment. In some embodiments, the reduction of depression is characterized by a decline in HAM-D value ranging from about one point to about twenty points, for example, about one point, about two points, about three points, about four points, about five points, about six points, about seven points, about eight points, about nine points, about ten points, about eleven points, about twelve points, about thirteen points, about fourteen points, about fifteen points, about sixteen points, about seventeen points, about eighteen points, about nineteen points, and about twenty points compared to prior to the treatment. In some embodiments, the reduction of depression is characterized by a decline HAM-D value of about two points. In some embodiments, the reduction of depression is characterized by a decline in HAM-D value of about three points. In some embodiments, the reduction of depression is characterized by a decline in HAM-D value of about four points. In some embodiments, the reduction of depression is characterized by a decline in HAM-D value of about five points. In some embodiments, the reduction of depression is characterized by a decline HAM-D value of about six points. In some embodiments, the reduction of depression is characterized by a decline in HAM-D value of about seven points. In some embodiments, the reduction of depression is characterized by a decline in HAM-D value of about eight points. In some embodiments, the reduction of depression is characterized by a decline in HAM-D value of about nine points. In some embodiments, the reduction of depression is characterized by a decline HAM-D value of about ten points. In some embodiments, the reduction of depression is characterized by a decline in HAM-D value of about eleven points. In some embodiments, the reduction of depression is characterized by a decline in HAM-D value of about twelve points. In some embodiments, the reduction of depression is characterized by a decline in HAM-D value of about thirteen points. In some embodiments, the reduction of depression is characterized by a decline HAM-D value of about fourteen points. In some embodiments, the reduction of depression is characterized by a decline in HAM-D value of about fifteen points. In some embodiments, the reduction of depression is characterized by a decline in HAM-D value of about sixteen points. In some embodiments, the reduction of depression is characterized by a decline in HAM-D value of about seventeen points. In some embodiments, the reduction of depression is characterized by a decline HAM-D value of about eighteen points. In some embodiments, the reduction of depression is characterized by a decline in HAM-D value of about nineteen points. In some embodiments, the reduction of depression is characterized by a decline in HAM-D value of about twenty points.

In some embodiments, after the treatment the patient experiences a substantial reduction of depression that is characterized by at least a one category change in HAM-D severity classification compared to prior to the treatment. In some embodiments, the reduction of depression is characterized by a one category change HAM-D severity classification compared to prior to the treatment. In some embodiments, the reduction of depression is characterized by a two category change HAM-D severity classification compared to prior to the treatment. In some embodiments, the reduction of depression is characterized by a three category change HAM-D severity classification compared to prior to the treatment. In some embodiments, the reduction of depression is characterized by remission according to HAM-D value after said treatment (i.e., total HAM-D value of 7 or less).

The Montgomery Asberg Depression Rating Scale (MADRS) is a depression rating scale consisting of 10 items, each rated 0 to 6. The 10 items represent the core symptoms of depressive illness. The total score of the 10 items ranges from 0 to 60. Decrease in the total score or on individual items indicates improvement (Montgomery S. A. and Asberg M. A, New Depression Scale Designed to be Sensitive to Change, Br. J. Psychiatry. (1979) April; 134, pages 382-9).

In some embodiments, after the treatment the patient experiences a substantial reduction of depression that is characterized by at least about a 30% decline in Montgomery Asberg Depression Rating Scale (MADRS) compared to prior to the treatment. In some embodiments, the reduction of depression is characterized by a decline in MADRS value ranging from about 30% to about 100%, for example, about 30%, about 40%, about 50%, about 60%, about 70%, about 80%, about 90%, and about 100%, compared to prior to the treatment.

In some embodiments, after the treatment the patient experiences a substantial reduction of depression that is characterized by at least a one point decline in MADRS value compared to prior to the treatment. In some embodiments, the reduction of depression is characterized by a decline in MADRS value ranging from about one point to about five points, for example, about one point, about two points, about three points, about four points, and about five points compared to prior to the treatment. In some embodiments, the reduction of depression is characterized by a decline MADRS value of about two points. In some embodiments, the reduction of depression is characterized by a decline in MADRS value of about three points. In some embodiments, the reduction of depression is characterized by a decline in MADRS value of about four points. In some embodiments, the reduction of depression is characterized by a decline in MADRS value of about five points. In some embodiments, the reduction of depression is characterized by remission according to MADRS value after said treatment (i.e., MADRS value of 12 or less).

The Hamilton Rating Scale for Anxiety (HAM-A) is an anxiety rating scale consisting of 14 items that assess anxious mood, tension, fear, insomnia, intellectual (cognitive) functioning, depressed mood, behavior at interview, somatic (sensory), cardiovascular, respiratory, gastrointestinal, genitourinary, autonomic, and somatic (muscular) symptom (Hamilton, M. The Assessment of Anxiety States by Rating, Br J Med Psychol. (1959); 32 (1), pages 50-5). Each symptom is rated from 0 (absent) to 4 (maximum severity) scale. The total score is used to categorize the severity of anxiety: mild severity (total score less than 17), mild to moderate severity (total score between 18-24), and moderate to severe (total score between 25-30). Total scores range from 0 to 56 with higher scores indicating greater severity.

In some embodiments, after the treatment the patient experiences a substantial reduction of depression that is characterized by at least about a 30% decline in total HAM-A value compared to prior to the treatment. In some embodiments, the reduction of depression is characterized by a decline in HAM-A value ranging from about 10% to about 100%, for example, about 20%, about 30%, about 40%, about 50%, about 60%, about 70%, about 80%, about 90%, and about 100%, compared to prior to the treatment.

In some embodiments, after the treatment the patient experiences a substantial reduction of depression that is characterized by at least a one point decline in HAM-A value compared to prior to the treatment. In some embodiments, the reduction of depression is characterized by a decline in HAM-A value ranging from about one point to about five points, for example, about one point, about two points, about three points, about four points, and about five points compared to prior to the treatment. In some embodiments, the reduction of depression is characterized by a decline HAM-A value of about two points. In some embodiments, the reduction of depression is characterized by a decline in HAM-A value of about three points. In some embodiments, the reduction of depression is characterized by a decline in HAM-A value of about four points. In some embodiments, the reduction of depression is characterized by a decline in HAM-A value of about five points.

In some embodiments, after the treatment the patient experiences a substantial reduction of depression that is characterized by at least a one category change in HAM-A severity classification compared to prior to the treatment. In some embodiments, the reduction of depression is characterized by a one category change HAM-A severity classification compared to prior to the treatment. In some embodiments, the reduction of depression is characterized by a two category change HAM-A severity classification compared to prior to the treatment. In some embodiments, the reduction of depression is characterized by a three category change HAM-A severity classification compared to prior to the treatment.

In some embodiments, after the treatment the patient experiences a substantial reduction of depression that is characterized by partial remission of the patient's depression. In some embodiments, after the treatment the patient experiences a substantial reduction of MDD that is characterized by partial remission of the patient's depression. In some embodiments, partial remission of MMD is where the symptoms of the immediately previous major depressive episode are present, but full criteria are not met, or there is a period lasting less than 2 months without any significant symptoms of a major depressive episode following the end of such an episode (i.e., the DSM-5's definition of partial remission).

In some embodiments, after the treatment the patient experiences a substantial reduction of depression that is characterized by full remission of the patient's depression. In some embodiments, after the treatment the patient experiences a substantial reduction of MDD that is characterized by full remission of the patient's depression. In some embodiments, full remission is where during the past 2 months, no significant signs or symptoms of the disturbance were present (i.e., the DSM-5's definition of full remission).

The Clinical Global Impression (CGI) (Guy 1976 (Guy W (1976), ECDEU Assessment Manual for Psychopharmacology, Revised. Rockville, Md.: US Department of Health, Education and Welfare) consists of three subscales: the CGI-Severity (CGI-S), the CGI-Improvement (CGI-I) and Efficacy Index. The CGI-S assesses the clinician's impression of the patient's current mental illness. A treating clinician categorizes the severity of the patient's current mental illness on a 7-point scale: 1 (normal, not at all ill), 2 (borderline mentally ill), 3 (mildly ill), 4 (moderately ill), 5 (markedly ill), 6 (severely ill), and 7 (among the most extremely ill patients). The CGI-I assesses the participant's improvement (or worsening) from baseline. A treating clinician categorizes the patient's condition relative to Baseline (e.g., prior to administering an antidepressant) on a 7-point scale: 1 (very much improved), 2 (much improved), 3 (minimally improved), 4 (no change), 5 (minimally worse), 6 (much worse), and 7 (very much worse).

In some embodiments, after the treatment the patient experiences a substantial reduction of depression that is characterized by at least a one point decline in CGI-S value compared to prior to the treatment. In some embodiments, the reduction of depression is characterized by a one point decline in CGI-S value compared to prior to the treatment. In some embodiments, the reduction of depression is characterized by a two point decline in CGI-S value compared to prior to the treatment. In some embodiments, the reduction of depression is characterized by a three point decline in CGI-S value compared to prior to the treatment.

In some embodiments, after the treatment the patient experiences a substantial reduction of depression that is characterized by at least a one point decline in CGI-I value compared to prior to the treatment. In some embodiments, the reduction of depression is characterized by a one point decline in CGI-I value compared to prior to the treatment. In some embodiments, the reduction of depression is characterized by a two point decline in CGI-I value compared to prior to the treatment. In some embodiments, the reduction of depression is characterized by a three point decline in CGI-I value compared to prior to the treatment.

The Symptoms of Depression Questionnaire (SDQ) is a 44-item, self-report scale that consists of five subscales: SDQ-1, SDQ-2, SDQ-3, SDQ-4 and SDQ-5. SDQ-1 includes items related to lassitude, mood, and cognitive functioning. SDQ-2 includes items related to anxiety, agitation, irritability, and anger. SDQ-3 includes items related to suicidal ideation. SDQ-4 assesses disruptions in sleep quality. SDQ-5 includes items on changes in appetite and weight. SDQ is used to assess symptom severity across several subtypes of depression (Pedrelli, P., et al., Reliability and Validity of the Symptoms of Depression Questionnaire (SDQ), CNS Spectr. 2014 December; 19(6), pages 535-546). The items are rated on a 6-point scale. Each item is rated based on a participant's perception of what is normal for the individual (score=2), what is better than normal (score=1), and what is worse than normal (scores=3-6). Total scores range from 0 to 264 with higher scores indicating greater severity.

In some embodiments, after the treatment the patient experiences a substantial reduction of depression that is characterized by at least about a 10% decline in total SDQ scale value compared to prior to the treatment. In some embodiments, the reduction of depression is characterized by a decline in total SDQ scale value ranging from about 10% to about 100%, for example, about 20%, about 30%, about 40%, about 50%, about 60%, about 70%, about 80%, about 90%, and about 100%, compared to prior to the treatment.

In some embodiments, after the treatment the patient experiences a substantial reduction of depression that is characterized by at least about a 10% decline in at least one subscale value selected from SDQ-1, SDQ-2, SDQ-3, SDQ-4 and SDQ-5 compared to prior to the treatment. In some embodiments, the reduction of depression is characterized by a decline in at least one subscale value selected from SDQ-1, SDQ-2, SDQ-3, SDQ-4 and SDQ-5 value ranging from about 10% to about 100%, for example, about 20%, about 30%, about 40%, about 50%, about 60%, about 70%, about 80%, about 90%, and about 100%, compared to prior to the treatment.

The Pittsburgh Sleep Quality Index (PSQI) is a 19-item self-report scale that assesses sleep quality and disturbances for the month preceding the assessment (Buysse D. J., The Pittsburgh Sleep Quality Index: a New Instrument for Psychiatric Practice and Research. Psychiatry Res. 1989 May; 28(2), pages 193-213). The scale generates seven “component” scores that differentiate “poor” from “good” sleep quality: subjective sleep quality, sleep latency, sleep duration, habitual sleep efficiency, sleep disturbances, use of sleeping medication, and daytime dysfunction. The sum of scores for these seven components yields the Global PSQI score. A Global PSQI score of “5” or greater indicates poor sleep quality.

In some embodiments, after the treatment the patient experiences a substantial reduction of depression that is characterized by at least a one point decline in Global PSQI score compared to prior to the treatment. In some embodiments, the reduction of depression is characterized by a one point decline in Global PSQI score compared to prior to the treatment. In some embodiments, the reduction of depression is characterized by a two point decline in Global PSQI score compared to prior to the treatment. In some embodiments, the reduction of depression is characterized by a three point decline in Global PSQI score compared to prior to the treatment.

In some embodiments, the method of treating depression further includes a step of titrating the dose of Compound 1 for at least about one week until a steady state is achieved in the patient. In one embodiment, the titration is conducted for about 2 weeks until a steady state is achieved in the patient. In another embodiment, the titration is conducted for about 7 days to about 30 days until a steady state is achieved in the patient. In another embodiment, the titration is conducted for about 12 days to about 20 days until a steady state is achieved in the patient. In some embodiments, during the titration step, a constant daily dose of Compound 1 is provided. In further embodiments, the constant daily dose of Compound 1 is provided for at least two weeks.

In some embodiments, ascending doses of Compound 1 are administered during the titration until a steady state is achieved in the patient. In some embodiments, ascending doses of the Compound 1 are administered during the titration until an effective amount of about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg or about 120 mg is achieved in the patient.

In some embodiments, the method of treating depression further includes a step of titrating the dose of ketamine until a therapeutically effective dose is achieved in the patient. In one embodiment, the titration is conducted for about 3 days until a therapeutically effective dose is achieved in the patient. In another embodiment, the titration is conducted for about 3 days to about 30 days, including about 6 days, about 9 days, about 12 days, about 15 days, about 18 days, about 21 days, about 24 days, about 27 and about 30 days, including all ranges in between, until a therapeutically effective dose is achieved in the patient. In some embodiments, the titration is conducted for about 3 days, about 6 days, about 9 days, about 12 days, about 15 days, about 18 days, about 21 days, about 24 days, about 27 or about 30 days until a therapeutically effective dose is achieved in the patient. In some embodiments, during the titration step, the daily dose of ketamine is increased about every three days until a therapeutically effective dose is achieved in the patient. In some embodiments, the daily dose of ketamine is increased by about 25 mg about every three days until a therapeutically effective dose is achieved in the patient.

In some embodiments, the present disclosure provides a method of treating depression that includes the steps of: (a) administering an initial dose of Compound 1 and ketamine for at least one week and (b) administering a maintenance dose of Compound 1 and ketamine for at least one week. In some embodiments, the initial dose is greater than the maintenance daily dose. In certain other embodiments, the initial dose is less than the maintenance daily dose. In some embodiments, the initial dose is administered for two weeks and the maintenance dose is administered is administered for at least one month.

In some embodiments, the initial dosing frequency and dose amount per administration of Compound 1 and ketamine are selected to provide therapeutic effects for the acute treatment of depression and the maintenance daily dosing frequency and dose amount per administration of Compound 1 and ketamine are selected to provide therapeutic effects for the chronic treatment of depression.

In some embodiments, the initial dosing frequency and dose amount per administration of Compound 1 and ketamine are selected to provide therapeutic effects for the acute treatment of depression and the maintenance daily dosing frequency and dose amount per administration of Compound 1 and ketamine are selected to maintain remission of depression.

In some embodiments, the initial dosing frequency and dose amount per administration of Compound 1 and ketamine are selected to provide therapeutic effects for the acute treatment of depression and the maintenance the daily dosing frequency and dose amount per administration of Compound 1 and ketamine are selected to prevent recurrence of depression.

In some embodiments, Compound 1 is initially dosed once a day. In some embodiments, Compound 1 is initially dosed twice a day. In some embodiments, the initial daily dose of Compound 1 is about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg or about 120 mg and the maintenance daily dose is about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, or about 115 mg provided the initial daily dose is greater than the maintenance daily dose.

In some embodiments, ketamine is initially dosed once a day. In some embodiments, ketamine is initially dosed twice a day. In some embodiments, ketamine is initially dosed once a week. In some embodiments, ketamine is initially dosed once a month. In some embodiments, the initial total daily (or total weekly or total monthly) dose of ketamine is about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, or about 250 mg provided the initial dose is greater than the maintenance dose.

According to some embodiments of the present invention, the methods of the present invention provide therapeutically effective blood plasma levels of Compound 1 and ketamine for treating depression. Blood plasma levels of Compound 1 and ketamine may be expressed using pharmacokinetic parameters that are known to those skilled in the art, such as steady state plasma levels, AUC, Cmax and Cmin.

In some embodiments, the present methods provide steady state plasma levels of Compound 1 that correlate to one or more statistically significant therapeutic effects. In some embodiments, the therapeutically effective steady state plasma levels of Compound 1 provided by the methods of the present invention range from about 1 ng/mL to about 200 ng/mL, including about 1 ng/ml, about 5 ng/mL, about 10 ng/mL, about 15 ng/mL, about 20 ng/mL, about 25 ng/mL, about 30 ng/mL, about 35 ng/mL, about 40 ng/mL, about 45 ng/mL, about 50 ng/mL, about 55 ng/mL, about 60 ng/mL, about 65 ng/mL, about 70 ng/mL, about 75 ng/mL about 80 ng/mL, about 85 ng/mL, about 90 ng/mL, about 95 ng/mL, about 100 ng/mL, about 105 ng/mL, about 110 ng/mL, about 115 ng/mL, about 120 ng/mL, about 125 ng/mL, about 130 ng/mL, about 135 ng/mL, about 140 ng/mL, about 145 ng/mL, about 150 ng/mL, about 155 ng/mL, about 160 ng/mL, about 165 ng/mL, about 170 ng/mL, about 175 ng/mL about 180 ng/mL, about 185 ng/mL, about 190 ng/mL, about 195 ng/mL, and 200 ng/ml, including all ranges there between. In some embodiments, the therapeutically effective steady state plasma levels of Compound 1 provided by the methods of the present invention range from about 50 ng/ml to 200 ng/ml.

In some embodiments, the therapeutically effective steady state plasma levels of Compound 1 is provided by administering a daily dose of Compound 1 or a pharmaceutically acceptable salt thereof of about 15 mg. In further embodiments, the therapeutically effective steady state plasma levels of Compound 1 is provided by administering about 15 mg of Compound 1 or a pharmaceutically acceptable salt thereof once a day.

In some embodiments, the therapeutically effective steady state plasma levels of Compound 1 is provided by administering a daily dose of Compound 1 or a pharmaceutically acceptable salt thereof of about 30 mg. In further embodiments, the therapeutically effective steady state plasma levels of Compound 1 is provided by administering about 15 mg of Compound 1 or a pharmaceutically acceptable salt thereof twice a day. In further embodiments, the therapeutically effective steady state plasma levels of Compound 1 is provided by administering about 30 mg of Compound 1 or a pharmaceutically acceptable salt thereof once a day.

In some embodiments, the therapeutically effective steady state plasma levels of Compound 1 is provided by administering a daily dose of Compound 1 or a pharmaceutically acceptable salt thereof of about 45 mg. In further embodiments, the therapeutically effective steady state plasma levels of Compound 1 is provided by administering about 45 mg of Compound 1 or a pharmaceutically acceptable salt thereof once a day.

In some embodiments, the therapeutically effective steady state plasma levels of Compound 1 is provided by administering a daily dose of Compound 1 or a pharmaceutically acceptable salt thereof of about 60 mg. In further embodiments, the therapeutically effective steady state plasma levels of Compound 1 is provided by administering about 30 mg of Compound 1 or a pharmaceutically acceptable salt thereof twice a day. In further embodiments, the therapeutically effective steady state plasma levels of Compound 1 is provided by administering about 60 mg of Compound 1 or a pharmaceutically acceptable salt thereof once a day.

In some embodiments, the present methods provide mean steady state AUC₀₋₂₄ h (expressed in terms of ng*hr/mL) levels of Compound 1 that correlate to one or more statistically significant therapeutic effects. In some embodiments, the therapeutically effective mean steady state AUC₀₋₂₄ h levels of Compound 1 provided by the methods of the present invention range from about 50 ng*hr/mL to about 2300 ng*hr/mL, including about 50 ng*hr/mL, about 100 ng*hr/mL, about 150 ng*hr/mL, about 200 ng*hr/mL, about 250 ng*hr/mL, about 300 ng*hr/mL, about 400 ng*hr/mL, about 500 ng*hr/mL, about 600 ng*hr/mL, about 700 ng*hr/mL, about 800 ng*hr/mL, about 900 ng*hr/mL, about 1000 ng*hr/mL, about 1100 ng*hr/mL, about 1200 ng*hr/mL, about 1300 ng*hr/mL, about 1400 ng*hr/mL, about 1500 ng*hr/mL, about 1600 ng*hr/mL, about 1700 ng*hr/mL, about 1800 ng*hr/mL, about 1900 ng*hr/mL, about 2000 ng*hr/mL, about 2100 ng*hr/mL, about 2200 ng*hr/mL and about 2300 ng*hr/mL, including all ranges there between.

In some embodiments, the mean steady state AUC₀₋₂₄ h levels of Compound 1 is provided by administering a daily dose of Compound 1 or a pharmaceutically acceptable salt thereof of about 15 mg. In further embodiments, the mean steady state AUC₀₋₂₄ h levels of Compound 1 is provided by administering about 15 mg of Compound 1 or a pharmaceutically acceptable salt thereof once a day.

In some embodiments, the mean steady state AUC₀₋₂₄ h levels of Compound 1 is provided by administering a daily dose of Compound 1 or a pharmaceutically acceptable salt thereof of about 30 mg. In further embodiments, the mean steady state AUC₀₋₂₄ h levels of Compound 1 is provided by administering about 15 mg of Compound 1 or a pharmaceutically acceptable salt thereof twice a day. In further embodiments, the mean steady state AUC₀₋₂₄ h levels of Compound 1 is provided by administering about 30 mg of Compound 1 or a pharmaceutically acceptable salt thereof once a day.

In some embodiments, the mean steady state AUC₀₋₂₄ h levels of Compound 1 is provided by administering a daily dose of Compound 1 or a pharmaceutically acceptable salt thereof of about 45 mg. In further embodiments, the mean steady state AUC₀₋₂₄ h levels of Compound 1 is provided by administering about 45 mg of Compound 1 or a pharmaceutically acceptable salt thereof once a day.

In some embodiments, the mean steady state AUC₀₋₂₄ h levels of Compound 1 is provided by administering a daily dose of Compound 1 or a pharmaceutically acceptable salt thereof of about 60 mg. In further embodiments, the mean steady state AUC₀₋₂₄ h levels of Compound 1 is provided by administering about 30 mg of Compound 1 or a pharmaceutically acceptable salt thereof twice a day. In further embodiments, the mean steady state AUC₀₋₂₄ h levels of Compound 1 is provided by administering about 60 mg of Compound 1 or a pharmaceutically acceptable salt thereof once a day.

In some embodiments, the present methods provide steady state plasma Cmax levels of Compound 1 that correlate to one or more statistically significant therapeutic effects. In some embodiments, the therapeutically effective steady state plasma Cmax levels of Compound 1 provided by the methods of the present invention range from about 5 ng/mL to about 500 ng/mL, including about 5 ng/mL, about 10 ng/mL, about 20 ng/mL, about 30 ng/mL, about 40 ng/mL, about 50 ng/mL, about 60 ng/mL, about 70 ng/mL, about 80 ng/mL, about 90 ng/mL, about 100 ng/mL, about 110 ng/mL, about 120 ng/mL, about 130 ng/mL, about 140 ng/mL, about 150 ng/mL, about 160 ng/mL, about 170 ng/mL about 180 ng/mL, about 190 ng/mL, about 200 ng/mL, about 210 ng/mL, about 220 ng/mL, about 230 ng/mL, about 240 ng/mL, about 250 ng/mL, about 260 ng/mL, about 270 ng/mL about 280 ng/mL, about 290 ng/mL, about 300 ng/mL, about 310 ng/mL, about 320 ng/mL, about 330 ng/mL, about 340 ng/mL, about 350 ng/mL, about 360 ng/mL, about 370 ng/mL about 380 ng/mL, about 390 ng/mL, about 400 ng/mL, about 410 ng/mL, about 420 ng/mL, about 430 ng/mL, about 440 ng/mL, about 150 ng/mL, about 460 ng/mL, about 470 ng/mL about 480 ng/mL, about 490 ng/mL, about 400 ng/mL, and about 500 ng/mL, including all ranges there between.

In some embodiments, the therapeutically effective steady state plasma Cmax levels of Compound 1 is provided by administering a daily dose of Compound 1 or a pharmaceutically acceptable salt thereof is about 15 mg. In further embodiments, the therapeutically effective steady state plasma Cmax levels of Compound 1 is provided by administering about 15 mg of Compound 1 or a pharmaceutically acceptable salt thereof once a day.

In some embodiments, the therapeutically effective steady state plasma Cmax levels of Compound 1 is provided by administering a daily dose of Compound 1 or a pharmaceutically acceptable salt thereof is about 30 mg. In further embodiments, the therapeutically effective steady state plasma Cmax levels of Compound 1 is provided by administering about 15 mg of Compound 1 or a pharmaceutically acceptable salt thereof twice a day. In further embodiments, the therapeutically effective steady state Cmax plasma levels of Compound 1 is provided by administering about 30 mg of Compound 1 or a pharmaceutically acceptable salt thereof once a day.

In some embodiments, the therapeutically effective steady state plasma Cmax levels of Compound 1 is provided by administering a daily dose of Compound 1 or a pharmaceutically acceptable salt thereof is about 45 mg. In further embodiments, the therapeutically effective steady state plasma Cmax levels of Compound 1 is provided by administering about 45 mg of Compound 1 or a pharmaceutically acceptable salt thereof once a day.

In some embodiments, the therapeutically effective steady state plasma Cmax levels of Compound 1 is provided by administering a daily dose of Compound 1 or a pharmaceutically acceptable salt thereof is about 60 mg. In further embodiments, the therapeutically effective steady state plasma Cmax of Compound 1 is provided by administering about 30 mg of Compound 1 or a pharmaceutically acceptable salt thereof twice a day. In further embodiments, the therapeutically effective steady state plasma Cmax levels of Compound 1 is provided by administering about 60 mg of Compound 1 or a pharmaceutically acceptable salt thereof once a day.

In some embodiments, the present methods provide steady state plasma Cmax levels of Compound 1 that do not exceed 500 ng/mL. In some embodiments, the therapeutically effective steady state plasma Cmax levels of Compound 1 provided by the methods of the present invention do not exceed about 500 ng/mL, including less than about 500 ng/mL, less than about 475 ng/mL, less than about 450 ng/mL, than about 425 ng/mL, less than about 400 ng/mL, less than about 375 ng/mL, than about 350 ng/mL, less than about 325 ng/mL, and less than about 300 ng/mL.

In some embodiments, the present methods provide mean steady state AUC₀₋₆ h (expressed in terms of ng*hr/mL) levels of ketamine that correlate to one or more statistically significant therapeutic effects. In some embodiments, the therapeutically effective mean steady state AUC₀₋₆ h levels of ketamine provided by the methods of the present invention range from about 50 ng*hr/mL to about 500 ng*hr/mL, including about 50 ng*hr/mL, about 75 ng*hr/mL, about 100 ng*hr/mL, about 125 ng*hr/mL, about 150 ng*hr/mL, about 175 ng*hr/mL, about 200 ng*hr/mL, about 200 ng*hr/mL, about 225 ng*hr/mL, about 250 ng*hr/mL, about 275 ng*hr/mL, 300 ng*hr/mL, about 320 ng*hr/mL, about 350 ng*hr/mL, about 375 ng*hr/mL, about 400 ng*hr/mL, about 425 ng*hr/mL, about 450 ng*hr/mL, about 475 ng*hr/mL, and about 500 ng*hr/mL, including all ranges there between.

In some embodiments, the present methods provide mean steady state AUC₀₋₆ h (expressed in terms of ng*hr/mL) levels of norketamine (a ketamine and esketamine metabolite) that correlate to one or more statistically significant therapeutic effects. In some embodiments, the therapeutically effective mean steady state AUC₀₋₆ h levels of norketamine provided by the methods of the present invention range from about 50 ng*hr/mL to about 500 ng*hr/mL, including about 50 ng*hr/mL, about 75 ng*hr/mL, about 100 ng*hr/mL, about 125 ng*hr/mL, about 150 ng*hr/mL, about 175 ng*hr/mL, about 200 ng*hr/mL, about 200 ng*hr/mL, about 225 ng*hr/mL, about 250 ng*hr/mL, about 275 ng*hr/mL, 300 ng*hr/mL, about 320 ng*hr/mL, about 350 ng*hr/mL, about 375 ng*hr/mL, about 400 ng*hr/mL, about 425 ng*hr/mL, about 450 ng*hr/mL, about 475 ng*hr/mL, and about 500 ng*hr/mL, including all ranges there between.

In some embodiments, the present methods provide steady state plasma Cmax levels of ketamine that correlate to one or more statistically significant therapeutic effects. In some embodiments, the therapeutically effective steady state plasma Cmax levels of ketamine provided by the methods of the present invention range from about 5 ng/mL to about 400 ng/mL, including about 5 ng/mL, about 10 ng/mL, about 20 ng/mL, about 30 ng/mL, about 40 ng/mL, about 50 ng/mL, about 60 ng/mL, about 70 ng/mL, about 80 ng/mL, about 90 ng/mL, about 100 ng/mL, about 110 ng/mL, about 120 ng/mL, about 130 ng/mL, about 140 ng/mL, about 150 ng/mL, about 160 ng/mL, about 170 ng/mL about 180 ng/mL, about 190 ng/mL, about 200 ng/mL, about 210 ng/mL, about 220 ng/mL, about 230 ng/mL, about 240 ng/mL, about 250 ng/mL, about 260 ng/mL, about 270 ng/mL about 280 ng/mL, about 290 ng/mL, about 300 ng/mL, about 310 ng/mL, about 320 ng/mL, about 330 ng/mL, about 340 ng/mL, about 350 ng/mL, about 360 ng/mL, about 370 ng/mL about 380 ng/mL, about 390 ng/mL, and about 400 ng/mL, including all ranges there between.

In some embodiments, the present methods provide steady state plasma Cmax levels of norketamine (a ketamine and esketamine metabolite) that correlate to one or more statistically significant therapeutic effects. In some embodiments, the therapeutically effective steady state plasma Cmax levels of norketamine provided by the methods of the present invention range from about 5 ng/mL to about 400 ng/mL, including about 5 ng/mL, about 10 ng/mL, about 20 ng/mL, about 30 ng/mL, about 40 ng/mL, about 50 ng/mL, about 60 ng/mL, about 70 ng/mL, about 80 ng/mL, about 90 ng/mL, about 100 ng/mL, about 110 ng/mL, about 120 ng/mL, about 130 ng/mL, about 140 ng/mL, about 150 ng/mL, about 160 ng/mL, about 170 ng/mL about 180 ng/mL, about 190 ng/mL, about 200 ng/mL, about 210 ng/mL, about 220 ng/mL, about 230 ng/mL, about 240 ng/mL, about 250 ng/mL, about 260 ng/mL, about 270 ng/mL about 280 ng/mL, about 290 ng/mL, about 300 ng/mL, about 310 ng/mL, about 320 ng/mL, about 330 ng/mL, about 340 ng/mL, about 350 ng/mL, about 360 ng/mL, about 370 ng/mL about 380 ng/mL, about 390 ng/mL, and about 400 ng/mL, including all ranges there between.

According to some embodiments of the present invention, the dosing frequency and dose amount per administration of the GABA-A PAM and the NMDA antagonist, NMDA Negative Allosteric Modulator or NMDA partial agonist are selected to provide therapeutic effects for the treatment of pain. In some embodiments, the dosing frequency and dose amount per administration of the GABA-A PAM and the NMDA antagonist, NMDA Negative Allosteric Modulator or NMDA partial agonist are selected to provide therapeutic effects for the treatment of pain selected from acute pain, chronic pain, neuropathic pain, or pain associated with a disease (e.g., cancer pain, neuropathic pain associated with diabetic peripheral neuropathy).

### EXAMPLES

The present invention is further illustrated by reference to the following Examples. However, it is noted that these Examples, like the embodiments described above, are illustrative and are not to be construed as restricting the scope of the invention in any way.

### Example 1

Studies are conducted to measure the combinatorial effects of Compound 1 and ketamine in the treatment of mood and/or affective disorders. In particular, the tail suspension test is used to evaluate antidepressant efficacy of Compound 1 and ketamine, individually and in combination. Each compound individually produces a reduction in immobility time in the tail suspension test in mice. Given that Compound 1 and ketamine exert antidepressant efficacy through distinct mechanisms of action, the effects of sub-maximal doses are additive. Because the two mechanisms are complementary in terms of excitatory/inhibitory balance within brain circuits relevant to mood as NMDA antagonism produces a reduction in excitatory input and GABA-A PAM produces an increase in inhibitory input, the combination of Compound 1 and ketamine has a greater effect on excitatory/inhibitory balance in the direction of inhibition. The result is that submaximal doses of both compounds which individually produce ˜10-15% reduction in immobility time, in combination, produce a greater, ˜30% reduction in immobility time, indicating a greater antidepressant effect. Given the acute and durable antidepressant effects of Compound 1 and ketamine, the effects of the combination are apparent both acutely after dosing and 24 h later.

Protocol: Adult male mice are separated into three groups and dosed with Compound 1, ketamine or vehicle control as described below:

Group 1: Compound 1 (0.1-10 mg/kg i.p.);

Group 2: Ketamine (0.3-30 mg/kg s.c.); and

Group 3: vehicle control.

Thirty minutes after dosing, the mice are suspended by their tails in individual chambers. The behavior of mice is monitored to determine the duration of immobility time during a 6 min session. An antidepressant effect is demonstrated when compounds produce a significant reduction in immobility time relative to vehicle control.

After determining the antidepressant effects of individual compounds, 2 additional cohorts of mice are co-administered a sub-maximal dose of ketamine (0.3-3 mg/kg s.c.) with a range of doses of Compound 1 (0.1-3 mg/kg i.p.). Effects of these combinations on immobility time are compared to the effects of the individual compounds to measure additive or synergistic effects: 1 cohort is assessed acutely, 30 minutes after dosing, a second is assessed 24 h after dosing.

### Example 2

Studies are conducted to measure the combinatorial effects of Compound 1 and ketamine in the treatment of mood and/or affective disorders. In particular, the forced swim test is used to evaluate antidepressant efficacy of Compound 1 and ketamine, individually and in combination. Each compound individually produces a reduction in immobility time in the forced swim test in mice. Given that Compound 1 and ketamine exert antidepressant efficacy through distinct mechanisms of action, the effects of sub-maximal doses are additive. Because the two mechanisms are complementary in terms of excitatory/inhibitory balance within brain circuits relevant to mood as NMDA antagonism produces a reduction in excitatory input and GABA-A PAM produces an increase in inhibitory input, the combination of Compound 1 and ketamine has a greater effect on excitatory/inhibitory balance in the direction of inhibition. The result is that submaximal doses of both compounds which individually produce ˜10-15% reduction in immobility time, in combination, produce a greater, ˜30% reduction in immobility time, indicating a greater antidepressant effect. Given the acute and durable antidepressant effects of Compound 1 and ketamine, the effects of the combination are apparent both acutely after dosing and 24 h later.

Protocol: Adult male mice are separated into three groups and dosed with Compound 1, ketamine or vehicle control as described below:

Group 1: Compound 1 (0.1-10 mg/kg i.p.);

Group 2: Ketamine (0.3-30 mg/kg s.c.); and

Group 3: vehicle control.

Thirty minutes after dosing, the mice are placed in individual beakers of water where they are unable to reach the bottom. Initially mice are very active as they try to get out of the water, but then become immobile, performing only the minimum movements required to stay afloat. The behavior of mice is monitored to determine the duration of immobility time during a 6 min session. An antidepressant effect is demonstrated when compounds produce a significant reduction in immobility time relative to vehicle control.

After determining the antidepressant effects of individual compounds, 2 additional cohorts of mice are co-administered a sub-maximal dose of ketamine (0.3-3 mg/kg s.c.) with a range of doses of Compound 1 (0.1-3 mg/kg i.p.). Effects of these combinations on immobility time are compared to the effects of the individual compounds to measure additive or synergistic effects: 1 cohort are assessed acutely, 30 minutes after dosing, a second is assessed 24 h after dosing.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as acknowledgment or any form of suggestion that they constitute valid prior art or form part of the common general knowledge in any country in the world.

### EMBODIMENTS

1. A composition for treating a mood or affective disorder comprising:
   a) a therapeutically effective amount of a GABA-A PAM and
   b) a therapeutically effective amount of an NMDA antagonist, NMDA open channel blocker, NMDA Negative Allosteric Modulator or NMDA partial agonist.
2. The composition of embodiment 1, wherein the GABA-A PAM is Compound 1:

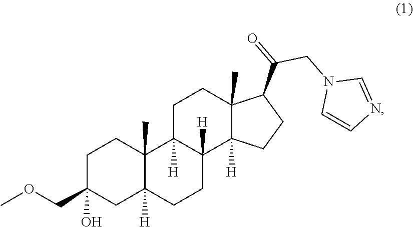

or a pharmaceutically acceptable salt thereof
3. The composition of any one of embodiments 1-2, wherein the NMDA antagonist is ketamine or a pharmaceutically acceptable salt thereof
4. The composition of any one of embodiments 1-2, wherein the NMDA antagonist is esketamine or a pharmaceutically acceptable salt thereof
5. The composition of any one of embodiments 2-4, wherein the pharmaceutically acceptable salt of Compound 1 is selected from the group consisting of hydrobromide, citrate, malate, mesylate, phosphate, and tartrate.
6. The composition of any one of embodiments 2-5, wherein upon administration to a patient in need thereof the composition provides a mean steady state blood plasma AUC (0-24) hours from about 500 ng*h/ml to about 2500 ng*h/ml of Compound 1.
7. The composition of any one of embodiments 2-6, wherein upon administration to a patient in need thereof the composition provides a steady state blood plasma Cmax from about 50 ng/mL to about 500 ng/ml of Compound 1.
8. The composition of any one of embodiments 2-7, wherein upon administration to a patient in need thereof the composition provides a steady state blood plasma Cmax that does not exceed 500 ng/ml of Compound 1.
9. The composition of any one of embodiments 2-8, wherein the composition is an oral dosage form.
10. The composition of any one of embodiments 2-8, wherein the composition is a nasal spray.
11. The composition of any one of embodiments 2-9, wherein the composition comprises from about 5 to about 120 mg of Compound 1.
12. The composition of any one of embodiments 2-9, wherein the composition comprises from about 1 mg to about 250 mg of ketamine or a pharmaceutically acceptable salt thereof
13. A kit for treating a mood or affective disorder comprising
   a) a composition comprising a therapeutically effective amount of a GABA-A PAM and
   b) a composition comprising a therapeutically effective amount of an NMDA antagonist, NMDA open channel blocker, NMDA Negative Allosteric Modulator or NMDA partial agonist.
14. The kit of embodiment 13, wherein the GABA-A PAM is Compound 1:

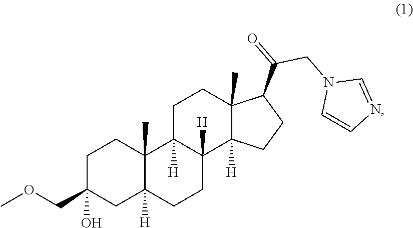

or a pharmaceutically acceptable salt thereof
15. The kit of any one of embodiments 13-14, wherein the NMDA antagonist is ketamine or a pharmaceutically acceptable salt thereof
16. The kit of any one of embodiments 13-14, wherein the NMDA antagonist is esketamine or a pharmaceutically acceptable salt thereof
17. The kit of any one of embodiments 14-16, wherein the pharmaceutically acceptable salt of Compound 1 is selected from the group consisting of hydrobromide, citrate, malate, mesylate, phosphate, and tartrate.
18. The kit of any one of embodiments 14-17, wherein upon administration to a patient in need thereof the composition (a) provides a mean steady state blood plasma AUC (0-24) hours from about 500 ng*h/ml to about 2500 ng*h/ml of Compound 1.
19. The kit of any one of embodiments 14-18, wherein upon administration to a patient in need thereof the composition (a) provides a steady state blood plasma Cmax from about 50 ng/mL to about 400 ng/ml of Compound 1.
20. The kit of any one of embodiments 14-19, wherein upon administration to a patient in need thereof the composition (a) provides a steady state blood plasma Cmax that does not exceed 500 ng/ml of Compound 1.
21. The kit of any one of embodiments 14-20, wherein the composition (a) is an oral dosage form.
22. The kit of any one of embodiments 14-20, wherein the composition (b) is an injectable dosage form.
23. The kit of any one of embodiments 14-22, wherein the composition (a) comprises from about 5 to about 120 mg of Compound 1.
24. The kit of any one of embodiments 14-21, wherein the composition (b) is an oral dosage form.
25. The kit of any one of embodiments 14-21, wherein the composition (b) is a nasal spray.
26. The kit of any one of embodiments 15 or 17-25, wherein the composition (b) comprises from about 1 to about 250 mg of ketamine or a pharmaceutically acceptable salt thereof
27. The kit of embodiment 25, wherein the composition (b) comprises esketamine or a pharmaceutically acceptable salt thereof
28. A method of treating a mood or affective disorder in a patient in need thereof comprising administering a therapeutically effective amount of a GABA-A PAM and a therapeutically effective amount of an NMDA antagonist, NMDA open channel blocker, NMDA Negative Allosteric Modulator or NMDA partial agonist.
29. The method of embodiment 28, wherein the mood or affective disorder is selected from the group consisting of perimenopause, generalized anxiety disorder, panic disorder, social anxiety disorder, post-traumatic stress disorder, specific phobia, and selective mutism.
30. The method of embodiment 28, wherein the mood or affective disorder is depression.
31. The method of embodiment 30, wherein the depression is selected from the group consisting of major depressive disorder, major depressive disorder with suicidal risk, clinical depression, postnatal or postpartum depression, treatment resistant postpartum depression, perimenopausal depression, premenstrual dysphoric disorder (PMDD), atypical depression, melancholic depression, Psychotic Major Depression (PMD), catatonic depression, Seasonal Affective Disorder (SAD), persistent depressive disorder (dysthymia), double depression, Depressive Personality Disorder (DPD), Recurrent Brief Depression (RBD), minor depressive disorder, bipolar disorder or manic depressive disorder, bipolar depression with suicidal risk, post-traumatic stress disorders, depression caused by chronic medical conditions, depressive disorder due to another medical condition, treatment-resistant depression, refractory depression, substance/medication induced depressive disorder, depression with anxiety, suicidality, suicidal ideation, or suicidal behavior.
32. The method of embodiment 31, wherein the depression is major depressive disorder.
33. The method of any one of embodiments 28-32, wherein the patient was previously treated with an NMDA antagonist but did not tolerate said treatment, and wherein the treated patient tolerates treatment with a GABA-A PAM and an NMDA antagonist.
34. The method of any one of embodiments 30-33, wherein after administering for a period of at least 1 week, the patient experiences a substantial reduction in depression compared to prior to said administering.
35. The method of any one of embodiments 30-34, wherein after administering for a period of at least 1 week, the patient experiences a reduction of depression that is characterized by an at least two point decline in total Hamilton Depression Rating Scale (HAM-D) value.
36. The method of any one of embodiments 30-34, wherein after administering for a period of at least 1 week, the patient experiences a reduction of depression that is characterized by an at least 50% reduction in HAM-D value.
37. The method of any one of embodiments 30-34, wherein after administering for a period of at least 1 week, the patient experiences a reduction of depression that is characterized by an at least one category change in HAM-D severity classification.
38. The method of any one of embodiments 30-34, wherein after administering for a period of at least 1 week, the patient experiences a reduction of depression that is characterized by HAM-D remission.
39. The method of any one of embodiments 30-34, wherein after administering for a period of at least 1 week, the patient experiences a reduction of depression that is characterized by an at least two point decline in Montgomery Asberg Depression Rating Scale (MADRS) value.
40. The method of any one of embodiments 30-34, wherein after administering for a period of at least 1 week, the patient experiences a reduction of depression that is characterized by an at least 50% reduction in MADRS value.
41. The method of any one of embodiments 30-34, wherein after administering for a period of at least 1 week, the patient experiences a reduction of depression that is characterized by MADRS remission.
42. The method of any one of embodiments 30-34, wherein after administering for a period of at least 1 week, the patient experiences a reduction of depression that is characterized by an at least two point decline in total Hamilton Rating Scale for anxiety (HAM-A) value.
43. The method of any one of embodiments 30-34, wherein after administering for a period of at least 1 week, the patient experiences a reduction of depression that is characterized by an at least 50% reduction in HAM-A value.
44. The method of any one of embodiments 30-34, wherein after administering for a period of at least 1 week, the patient experiences a reduction of depression that is characterized by an at least one category change in HAM-A severity classification.
45. The method of any one of embodiments 30-34, wherein after administering for a period of at least 1 week, the patient experiences a reduction of depression that is characterized by at least one point decline in one or more of the Clinical Global Impression (CGI) subscale scores, wherein the CGI subscales are selected from Severity of Illness Subscale (CGI-S) or Global Improvement Subscale (CGI-I).
46. The method of any one of embodiments 30-34, wherein after administering for a period of at least 1 week, the patient experiences a reduction of depression that is characterized by at least about a 10%, 20%, or 30% improvement in Symptoms of Depression Questionnaire (SDQ) total scale score or in any of the respective subscales of SDQ-1, SDQ-2, SDQ-3, SDQ-4 and SDQ-5.
47. The method of any one of embodiments 30-34, wherein after administering for a period of at least 1 week, the patient experiences a reduction of depression that is characterized by an at least one point decline in Pittsburgh Sleep Quality Index (PSQI) Global score.
48. The method of any one of embodiments 28-47, wherein the GABA-A PAM is selected from the group consisting of Compound 1 or a pharmaceutically acceptable salt thereof, gaboxadol, etifoxine, midazolam, diazepam, chlordiazepoxide, alprazolam, adinazolam pregnanolone, allopregnanolone, allotetrahydrodeoxycorticosterone, ganaxolone, alphaxolone, alphadolone, hydroxydione, minaxolone, Althesin, Renanolone, PF-06372865 (7-ethyl-4-[3-(4-ethylsulfonyl-2-methoxyphenyl)-4-fluorophenyl]imidazo[4,5-c]pyridazine), SAGE-324, SAGE-217 (3α-hydroxy-3β-methyl-21-(4-cyano-1H-pyrazol-1′-yl)-19-nor-5β-pregnan-20-one), and any neuroactive steroid as described in US2017/0240589 (herein incorporated by reference for all purposes).
49. The method of any one of embodiments 28-47, wherein the GABA-A PAM is Compound 1 or a pharmaceutically acceptable salt thereof
50. The method of any one of embodiment 28-49, wherein the NMDA antagonist, NMDA Negative Allosteric Modulator or NMDA partial agonist is selected from the group consisting of ketamine, R-ketamine, esketamine, methadone, D-methadone, BMT-108908 ((R)-1-(4-fluorobenzyl)-3-(4-(4-hydroxyphenyl)piperidin-1-yl)pyrrolidin-2-one), flupirtine radiprodil, priralfinamide dextromethorphan, AZD-8108, AZD-6423, NYX-2925 (((2S, 3R)-3-hydroxy-2-((R)-5-isobutyryl-1-oxo-2,5-diazaspiro[3.4]octan-2-yl)butanamide)), NYX-783, NYX-458, lanicemine, AZD 6423, traxoprodil, ifenprodil, rislenemdaz (CERC-301), EVT-101 (5-(3-(difluoromethyl)-4-fluorophenyl)-3-((2-methyl-1H-imidazol-1-yl)methyl)pyridazine), 4-chlorokynurenine, memantine, amantadine, methoxetamine, Rapastinel, AGN-241751, d-cycloserine, dextropropoxyphene, ketobemidone, phencyclidine, and riluzole.
51. The method of any one of embodiments 28-50, wherein the NMDA antagonist is ketamine or a pharmaceutically acceptable salt thereof
52. The method of any one of embodiments 28-50, wherein the NMDA antagonist is ketamine or a pharmaceutically acceptable salt thereof and the GABA-A PAM is Compound 1 or a pharmaceutically acceptable salt thereof
53. The method of any one of embodiments 28-50, wherein the NMDA antagonist is esketamine or a pharmaceutically acceptable salt thereof
54. The method of any one of embodiments 28-50, wherein the NMDA antagonist is esketamine or a pharmaceutically acceptable salt thereof and the GABA-A PAM is Compound 1 or a pharmaceutically acceptable salt thereof
55. The method of any one of embodiments 28-54, wherein the NMDA antagonist, NMDA open channel blocker, NMDA Negative Allosteric Modulator or NMDA partial agonist and the GABA-A PAM are administered sequentially.
56. The method of any one of embodiments 28-55, wherein the NMDA antagonist, NMDA open channel blocker, NMDA Negative Allosteric Modulator or NMDA partial agonist is administered in the morning and the GABA-A PAM is administered in the evening.
57. The method of any one of embodiments 28-54, wherein the NMDA antagonist, NMDA open channel blocker, NMDA Negative Allosteric Modulator or NMDA partial agonist and the GABA-A PAM are administered concomitantly.
58. The method of any one of embodiments 28-54, wherein the NMDA antagonist, NMDA open channel blocker, NMDA Negative Allosteric Modulator or NMDA partial agonist and the GABA-A PAM are administered in the same composition.
59. The method of any one of embodiments 28-58, wherein the GABA-A PAM is administered once per day.
60. The method of any one of embodiments 28-58, wherein the GABA-A PAM is administered twice per day.
61. The method of any one of embodiments 28-60, wherein the NMDA antagonist, NMDA open channel blocker, NMDA Negative Allosteric Modulator or NMDA partial agonist is administered about once a week.
62. The method of any one of embodiments 28-60, wherein the NMDA antagonist, NMDA open channel blocker, NMDA Negative Allosteric Modulator or NMDA partial agonist is administered about once every two weeks.
63. The method of any one of embodiments 28-60, wherein the NMDA antagonist, NMDA open channel blocker, NMDA Negative Allosteric Modulator or NMDA partial agonist is administered about once a month.
64. The method of any one of embodiments 28-63, wherein the administering of the GABA-A PAM increases the mean steady state blood plasma AUC₀₋₂₄ of the NMDA antagonist, NMDA Negative Allosteric Modulator or NMDA partial agonist compared to the mean steady state blood plasma AUC₀₋₂₄ when the same dose of the NMDA antagonist, NMDA Negative Allosteric Modulator or NMDA partial agonist is administered without the GABA-A PAM.
65. The method of embodiment 64, further comprising reducing the dose of the NMDA antagonist, NMDA open channel blocker, NMDA Negative Allosteric Modulator or NMDA partial agonist to provide a therapeutically effective plasma level of the NMDA antagonist, NMDA Negative Allosteric Modulator or NMDA partial agonist.
66. The method of any one of embodiments 28-65, wherein the therapeutically effective dose of GABA-A PAM and the NMDA antagonist, NMDA open channel blocker, NMDA Negative Allosteric Modulator or NMDA partial agonist is about 25% less than the therapeutically effective dose of GABA-A PAM and the NMDA antagonist, NMDA open channel blocker, NMDA Negative Allosteric Modulator or NMDA partial agonist when the GABA-A PAM and NMDA antagonist, NMDA open channel blocker, NMDA Negative Allosteric Modulator or NMDA partial agonist are not co-administered.
67. The method of any one of embodiments 28-53 or 59-66, wherein the NMDA antagonist, NMDA open channel blocker, NMDA Negative Allosteric Modulator or NMDA partial agonist is administered as an injectable dosage form.
68. The method of any one of embodiments 28-53 or 59-66, wherein the NMDA antagonist, NMDA open channel blocker, NMDA Negative Allosteric Modulator or NMDA partial agonist is administered in an oral dosage form.
69. The method of any one of embodiments 28-53 or 59-66, wherein the NMDA antagonist, NMDA open channel blocker, NMDA Negative Allosteric Modulator or NMDA partial agonist is administered as a nasal spray.
70. The method of embodiment 69, wherein the NMDA antagonist is esketamine and about 54 mg of esketamine or a pharmaceutically acceptable salt thereof is administered once a week or twice a week.
71. The method of embodiment 69, wherein the NMDA antagonist is esketamine and about 84 mg of esketamine or a pharmaceutically acceptable salt thereof is administered once a week or twice a week.
72. The method of embodiment of any of embodiments 28-71, wherein the method comprises administering a therapeutically effective amount of the composition of any one of embodiments 1-12 or the kit of any one of embodiments 13-27.
73. A method of treating adjustment disorder in a patient in need thereof comprising administering a therapeutically effective amount of a GABA-A PAM and a therapeutically effective amount of an NMDA antagonist, NMDA open channel blocker, NMDA Negative Allosteric Modulator or NMDA partial agonist.
74. The method of embodiment 73, wherein the adjustment disorder is adjustment disorder with depressed mood.
75. The method of embodiment 73, wherein the adjustment disorder is adjustment disorder with anxiety.
76. The method of embodiment 73, wherein the adjustment disorder is adjustment disorder with mixed anxiety and depressed mood.

1. A composition for treating a mood or affective disorder comprising:
a) a therapeutically effective amount of a GABA-A PAM and
b) a therapeutically effective amount of an NMDA antagonist, NMDA open channel blocker, NMDA Negative Allosteric Modulator or NMDA partial agonist.

2. The composition of claim 1, wherein the GABA-A PAM is Compound 1: 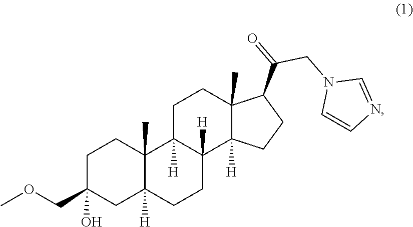
or a pharmaceutically acceptable salt thereof.

3. The composition of claim 1, wherein the NMDA antagonist is ketamine or a pharmaceutically acceptable salt thereof.

4. The composition of claim 1, wherein the NMDA antagonist is esketamine or a pharmaceutically acceptable salt thereof.

5. The composition of claim 2, wherein the pharmaceutically acceptable salt of Compound 1 is selected from the group consisting of hydrobromide, citrate, malate, mesylate, phosphate, and tartrate.

6. The composition of claim 2, wherein upon administration to a patient in need thereof the composition provides a mean steady state blood plasma AUC (0-24) hours from about 500 ng*h/ml to about 2500 ng*h/ml of Compound 1.

7. The composition of claim 2, wherein upon administration to a patient in need thereof the composition provides a steady state blood plasma Cmax from about 50 ng/mL to about 500 ng/ml of Compound 1.

8. The composition of claim 2, wherein upon administration to a patient in need thereof the composition provides a steady state blood plasma Cmax that does not exceed 500 ng/ml of Compound 1.

9. The composition of claim 2, wherein the composition is an oral dosage form.

10. The composition of claim 2, wherein the composition is a nasal spray.

11. The composition of claim 2, wherein the composition comprises from about 5 mg to about 120 mg of Compound 1.

12. The composition of claim 3, wherein the composition comprises from about 1 mg to about 250 mg of ketamine or a pharmaceutically acceptable salt thereof.

13. A kit for treating a mood or affective disorder comprising
a) a composition comprising a therapeutically effective amount of a GABA-A PAM and
b) a composition comprising a therapeutically effective amount of an NMDA antagonist, NMDA open channel blocker, NMDA Negative Allosteric Modulator or NMDA partial agonist.

14. The kit of claim 13, wherein the GABA-A PAM is Compound 1: 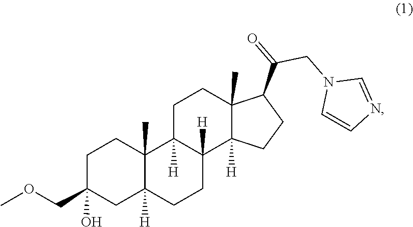
or a pharmaceutically acceptable salt thereof

15. The kit of claim 13, wherein the NMDA antagonist is ketamine or esketamine, or a pharmaceutically acceptable salt thereof.

16. (canceled)

17. The kit of claim 14, wherein the pharmaceutically acceptable salt of Compound 1 is selected from the group consisting of hydrobromide, citrate, malate, mesylate, phosphate, and tartrate.

18. The kit of claim 14, wherein upon administration to a patient in need thereof the composition (a) provides a mean steady state blood plasma AUC (0-24) hours from about 500 ng*h/ml to about 2500 ng*h/ml of Compound 1.

19. The kit of claim 14, wherein upon administration to a patient in need thereof the composition (a) provides a steady state blood plasma Cmax from about 50 ng/mL to about 400 ng/ml of Compound 1.

20. The kit of claim 14, wherein upon administration to a patient in need thereof the composition (a) provides a steady state blood plasma Cmax that does not exceed 500 ng/ml of Compound 1.

21. The kit of claim 14, wherein the composition (a) is an oral dosage form.

22. The kit of claim 14, wherein the composition (b) is an injectable dosage form, an oral dosage form, or a nasal spray.

23. The kit of claim 14, wherein the composition (a) comprises from about 5 to about 120 mg of Compound 1.

24-25. (canceled)

26. The kit of claim 15, wherein the composition (b) comprises from about 1 to about 250 mg of ketamine or a pharmaceutically acceptable salt thereof.

27. The kit of claim 22, wherein the composition (b) is a nasal spray and comprises esketamine or a pharmaceutically acceptable salt thereof.

28. A method of treating adjustment disorder in a patient in need thereof comprising administering a therapeutically effective amount of a GABA-A PAM and a therapeutically effective amount of an NMDA antagonist, NMDA open channel blocker, NMDA Negative Allosteric Modulator or NMDA partial agonist.
